(19) United States
(12) Patent Application Publication
(10) Pub. No.: US 2025/0179172 A1
(43) Pub. Date: Jun. 5, 2025

(54) CD5 ANTIBODY AND USE THEREOF

(71) Applicant: HAINAN SIMCERE ZAIMING PHARMACEUTICAL CO., LTD., Haikou, Hainan (CN)

(72) Inventors: Yulan HU, Shanghai (CN); Cuiqing YANG, Shanghai (CN); Zhuoxiao CAO, Shanghai (CN); Renhong TANG, Shanghai (CN); Jinsheng REN

(73) Assignee: HAINAN SIMCERE ZAIMING PHARMACEUTICAL CO., LTD., Haikou, Hainan (CN)

(21) Appl. No.: 18/267,518

(22) PCT Filed: Dec. 16, 2021
(86) PCT No.: PCT/CN2021/138649

§ 371 Date: Jun. 15, 2023

(30) Foreign Application Priority Data
Dec. 17, 2020 (CN) 202011490219.6

(51) Int. Cl. C07K 16/28

(52) U.S. Cl. CPC C07K 16/2803; C07K 2317/33; C07K 2317/52; C07K 2317/569; C07K 2317/92

(57) ABSTRACT

An antibody or antigen binding fragment that specifically binds to CD5, a polypeptide, a chimeric antigen receptor, an immune effector cell, a nucleic acid fragment, a vector, a host cell, a pharmaceutical composition, a preparation method, and use thereof in the treatment of diseases and in the detection of CD5, etc., which have important meanings for developing therapeutic drugs and CD5 detection reagents.

### CROSS-REFERENCE TO RELATED APPLICATIONS

This application is a National Phase application under 35 U.S.C. § 371 of International Patent Application No. PCT/CN2021/138649, titled “CD5 ANTIBODY AND USE THEREOF”, filed on Dec. 16, 2021, which claims the priority to Chinese Patent Application No. 202011490219.6, titled “CD5 ANTIBODY AND USE THEREOF”, filed on Dec. 17, 2020, with the China National Intellectual Property Administration, which are incorporated herein by reference in entireties.

### INCORPORATION OF SEQUENCE LISTING

The sequence listing that is contained in the file named “UNIT.P0106US_Corrected_Sequence_Listing”, which is 51 KB (as measured in Microsoft Windows®) and was created on Nov. 16, 2023, is filed herewith by electronic submission, and is incorporated by reference herein.

### TECHNICAL FIELD

The present disclosure relates to the field of antibodies, in particular to a CD5 antibody and use thereof.

### BACKGROUND

CD5 molecule is a 67-kDa type I transmembrane glycoprotein monomer, a kind of cell differentiation antigen and belongs to the scavenger receptor cysteine-rich (SRCR) superfamily (Sarrias M R, et al. Crit Rev Immunol. 2004. 24 (1): 1-37). CD5 contains three extracellular SRCR domains (D1, D2, D3) composed of 347 amino acids, a hydrophobic transmembrane domain and a large cytoplasmic region containing 93 amino acids but lacking intrinsic catalytic activity, and further contains several highly conserved elements which participate in signal transduction and possibly mediate protein-protein interaction, as well as potential phosphorylation sites (Ishida, et al. J Exp Med. 1992 May 1; 175 (5): 1213-20; Lozano et al. Crit Rev Immunol. 2000; 20 (4): 347-58). In fully processed mature CD5 protein, major signal motifs are located nearby the target of tyrosine kinase (Lck)-tyrosine residues Y429 and Y463 (J M Vilà, et al. Eur J Immunol. 2001 April; 31 (4): 1191-8.), and the target of casein kinase II (CKII)-serine residues S459 and S461 (Calvo et al. Tissue Antigens. 54:16-26. 1999). Human CD5 gene was cloned in 1986 (Jones et al. Nature. 1986. 323 (6086): 346-9). The upstream sequence of CD5 gene lacks a consensus TATA box, but contains regulatory elements such as IgM muE2, SV40, AP-1, SP-1, CCAAT, TCF2a/ets, PEA3 and c/EBP; the sequence from −125 bp to −27 bp (which contains Inr, IgM muE2, SV40 and AP-1), is the sufficient tissue-specific promoter sequence of the CD5 gene (Weichert T R, et al. J Immunol. 1995. 154 (9): 4603-12). CD5 mainly exists in mature peripheral T lymphocytes, also expressed in B1a cells of normal human lymphoid tissues. CD5 is correlated to the antigen-specific receptor existing in T cells and B cells and thus, can well regulate the signal reaction necessary for the activation and differentiation of T cells and B cells.

Main functions of CD5 is to negatively regulate T-cell receptor (TCR) signals and B-cell receptor (BCR) signals by activating negative regulatory factors such as CKII (Raman et al. 1998. J Biol Chem. 273:19183-19189) or Src homology region 2 domain-containing phosphatase SHP-1 (Perez-Villar et al. 1999. Mol Cell Biol. 19:2903-2912). In CD5+ B cells, CD5 prevents the apoptosis of B cells by down-regulating the BCR-mediated signal transduction pathway and accelerates the production of IL-10, thus providing survival factors for B cells. However, recent studies have shown that CD5 has broader functions, including regulation in the survival of lymphocytes, regulation in peripheral tolerance and as a pathogen-associated molecular pattern receptor (Soldevila et al. 2011. Curr Opin Immunol. 23: 310-318). As a pattern recognition receptor, CD5 senses the presence of conserved fungal components (namely, beta-glucans) instead of bacterial cell wall constituents (Vera et al. 2009. Proc Natl Acad Sci USA. 106:1506-1511). Evidences from an experimental autoimmune and tumor mouse model have indicated that CD5 accelerates the survival of T cells by down-regulating activation induced cell death (AICD) (Axtell et al. 2004. J Immunol. 173:2928-2932; Friedlein et al. 2007. J Immunol. 178:6821-6827.). The up-regulated expression of CD5 has been also reported as the feature of the regulatory lymphocytes (Treg and B10 cell) (Yanaba et al. 2008. Immunity 28:639-650; Ordonez-Rueda et al. 2009. Eur J Immunol. 39:2233-2247) and activated T and B cells (Hippen et al. 2000. J Exp Med. 191:883-890; Stamou et al. 2003. J Immunol. 171:1278-1284). All the reports have shown that CD5 is a correlated immunoreceptor under physiological and pathological conditions derived from autoimmunity, tumor or infection (Soldevila et al. 2011. Curr Opin Immunol. 23:310⁻³¹⁸).

In 1980, CD5 molecule was found by Royston, Wang (J Exp Med. 1992 May 1; 175 (5): 1213-20), et al., on the surface of peripheral B cells of a patient with chronic lymphocytic leukemia (CML). CD5 molecule is a characteristic surface marker to most of lymphocytic tumors, including T-cell acute lymphocytic leukemia (T-ALL), chronic lymphocytic leukemia (CLL), T-cell lymphoma and partial B-cell lymphomas; moreover, and CD5 is not expressed in normal hematopoietic stem cells and other types of non-hematopoietic cells. Therefore, as a tumor antigen with high specificity in lymphocytes, CD5 can serve as a desired therapeutic target in malignant tumors of lymphocytes.

In 1993, Hamers-Casterman C, a Belgian scientist, found a natural heavy chain antibody including a heavy chain but excluding a light chain in camel blood (Hamers-Casterman C et al. Nature. 363 (6428): 446-8 (1993).). Compared with conventional antibodies, the heavy chain antibody still keeps the antigen-binding capacity even though lacks the light chain. The variable region of the heavy chain antibody in camel body was cloned to obtain a single-domain antibody (sdAb) consisting of a heavy chain variable region only; the single-domain antibody is called a nanobody or a VHH antibody (variable heavy chain domain of a heavy chain antibody). Nanobody has the advantages such as small molecular weight, penetrability to blood brain barrier, high expression in prokaryotic or eukaryotic system, strong specificity, high affinity and weak immunogenicity to human. The specific structure of the VHH single-domain antibody based on the Vicugna pacos heavy chain antibody has the advantages of both conventional antibodies and small molecule drugs; moreover, the specific structure almost overcomes the defects of conventional antibodies, such as long development period, lower stability and demanding preservation conditions perfectly. Therefore, the specific structure has gradually become a newly emerging force in the new generation of therapeutic biomedicines and reagents for clinical diagnosis. Therefore, the research and development of a CD5 therapeutic nanobody have broad prospects.

### SUMMARY

The present disclosure provides an antibody or an antigen-binding fragment specifically binding to CD5, a polypeptide, a chimeric antigen receptor, an immune effector cell, a nucleic acid fragment, a vector, a host cell, a pharmaceutical composition, a preparation method, and use thereof in disease treatment, CD5 assay and other aspects.

In a first aspect, the present disclosure provides an antibody or an antigen-binding fragment specifically binding to CD5, wherein the antibody or the antigen-binding fragment comprises a CDR1, a CDR2, and a CDR3; the CDR1, the CDR2, and the CDR3 comprise an HCDR1, an HCDR2, and an HCDR3 selected from a VHH domain set forth in any one of SEQ ID NOs: 6-16.

In some specific embodiments, the HCDR1, the HCDR2, and the HCDR3 are determined according to the IMGT numbering scheme, the Kabat numbering scheme, or the Chothia numbering scheme; optionally, the HCDR1, the HCDR2, and the HCDR3 are selected from Table 3;
optionally, the HCDR1 has an amino acid sequence set forth in SEQ ID NO: 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, or 113;
optionally, the HCDR2 has an amino acid sequence set forth in SEQ ID NO: 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, or 114;
optionally, the HCDR3 has an amino acid sequence set forth in SEQ ID NO: 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, or 115;
preferably, the HCDR1, the HCDR2, and the HCDR3 have amino acid sequences set forth in SEQ ID NOs: 17-19 according to the IMGT numbering scheme; or
preferably, the HCDR1, the HCDR2, and the HCDR3 have amino acid sequences set forth in SEQ ID NOs: 20-22 according to the Kabat numbering scheme; or
preferably, the HCDR1, the HCDR2, and the HCDR3 have amino acid sequences set forth in SEQ ID NOs: 23-25 according to the Chothia numbering scheme; or
preferably, the HCDR1, the HCDR2, and the HCDR3 have amino acid sequences set forth in SEQ ID NOs: 26-28 according to the IMGT numbering scheme;
preferably, the HCDR1, the HCDR2, and the HCDR3 have amino acid sequences set forth in SEQ ID NOs: 29-31 according to the Kabat numbering scheme;
preferably, the HCDR1, the HCDR2, and the HCDR3 have amino acid sequences set forth in SEQ ID NOs: 32-34 according to the Chothia numbering scheme;
preferably, the HCDR1, the HCDR2, and the HCDR3 have amino acid sequences set forth in SEQ ID NOs: 35-37 according to the IMGT numbering scheme;
preferably, the HCDR1, the HCDR2, and the HCDR3 have amino acid sequences set forth in SEQ ID NOs: 38-40 according to the Kabat numbering scheme;
preferably, the HCDR1, the HCDR2, and the HCDR3 have amino acid sequences set forth in SEQ ID NOs: 41-43 according to the Chothia numbering scheme;
preferably, the HCDR1, the HCDR2, and the HCDR3 have amino acid sequences set forth in SEQ ID NOs: 44-46 according to the IMGT numbering scheme;
preferably, the HCDR1, the HCDR2, and the HCDR3 have amino acid sequences set forth in SEQ ID NOs: 47-49 according to the Kabat numbering scheme;
preferably, the HCDR1, the HCDR2, and the HCDR3 have amino acid sequences set forth in SEQ ID NOs: 50-52 according to the Chothia numbering scheme;
preferably, the HCDR1, the HCDR2, and the HCDR3 have amino acid sequences set forth in SEQ ID NOs: 53-55 according to the IMGT numbering scheme;
preferably, the HCDR1, the HCDR2, and the HCDR3 have amino acid sequences set forth in SEQ ID NOs: 56-58 according to the Kabat numbering scheme;
preferably, the HCDR1, the HCDR2, and the HCDR3 have amino acid sequences set forth in SEQ ID NOs: 59-61 according to the Chothia numbering scheme;
preferably, the HCDR1, the HCDR2, and the HCDR3 have amino acid sequences set forth in SEQ ID NOs: 62-64 according to the IMGT numbering scheme;
preferably, the HCDR1, the HCDR2, and the HCDR3 have amino acid sequences set forth in SEQ ID NOs: 65-67 according to the Kabat numbering scheme;
preferably, the HCDR1, the HCDR2, and the HCDR3 have amino acid sequences set forth in SEQ ID NOs: 68-70 according to the Chothia numbering scheme;
preferably, the HCDR1, the HCDR2, and the HCDR3 have amino acid sequences set forth in SEQ ID NOs: 71-73 according to the IMGT numbering scheme;
preferably, the HCDR1, the HCDR2, and the HCDR3 have amino acid sequences set forth in SEQ ID NOs: 74-76 according to the Kabat numbering scheme;
preferably, the HCDR1, the HCDR2, and the HCDR3 have amino acid sequences set forth in SEQ ID NOs: 77-79 according to the Chothia numbering scheme;
preferably, the HCDR1, the HCDR2, and the HCDR3 have amino acid sequences set forth in SEQ ID NOs: 80-82 according to the IMGT numbering scheme;
preferably, the HCDR1, the HCDR2, and the HCDR3 have amino acid sequences set forth in SEQ ID NOs: 83-85 according to the Kabat numbering scheme;
preferably, the HCDR1, the HCDR2, and the HCDR3 have amino acid sequences set forth in SEQ ID NOs: 86-88 according to the Chothia numbering scheme;
preferably, the HCDR1, the HCDR2, and the HCDR3 have amino acid sequences set forth in SEQ ID NOs: 89-91 according to the IMGT numbering scheme;
preferably, the HCDR1, the HCDR2, and the HCDR3 have amino acid sequences set forth in SEQ ID NOs: 92-94 according to the Kabat numbering scheme;
preferably, the HCDR1, the HCDR2, and the HCDR3 have amino acid sequences set forth in SEQ ID NOs: 95-97 according to the Chothia numbering scheme;
preferably, the HCDR1, the HCDR2, and the HCDR3 have amino acid sequences set forth in SEQ ID NOs: 98-100 according to the IMGT numbering scheme;
preferably, the HCDR1, the HCDR2, and the HCDR3 have amino acid sequences set forth in SEQ ID NOs: 101-103 according to the Kabat numbering scheme;
preferably, the HCDR1, the HCDR2, and the HCDR3 have amino acid sequences set forth in SEQ ID NOs: 104-106 according to the Chothia numbering scheme;
preferably, the HCDR1, the HCDR2, and the HCDR3 have amino acid sequences set forth in SEQ ID NOs: 107-109 according to the IMGT numbering scheme;
preferably, the HCDR1, the HCDR2, and the HCDR3 have amino acid sequences set forth in SEQ ID NOs: 110-112 according to the Kabat numbering scheme;
preferably, the HCDR1, the HCDR2, and the HCDR3 have amino acid sequences set forth in SEQ ID NOs: 113-115 according to the Chothia numbering scheme.

In some specific embodiments, the CDR1, the CDR2, and/or the CDR3 comprise amino acid sequences having at most 10, 9, 8, 7, 6, 5, 4, 3, 2, or 1 mutation on the HCDR1, the HCDR2, and/or the HCDR3; the mutation may be selected from an insertion, a deletion, and/or a substitution; the substitution is preferably a substitution of conserved amino acids.

In some specific embodiments, the CDR1, the CDR2, and/or the CDR3 comprise sequences having at least 80%, 85%, 90%, 91%, 92%, 93%, 94%, 95%, 96%, 97%, 98%, 99%, or 100% identity to the HCDR1, the HCDR2, and/or the HCDR3, respectively.

In some specific embodiments, the antibody or the antigen-binding fragment comprises a single-domain antibody comprising the CDR1, the CDR2, and the CDR3.

In some specific embodiments, the single-domain antibody comprises a sequence set forth in any one of SEQ ID NOs: 6-16;
optionally, the single-domain antibody comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 6-16; the mutation may be selected from an insertion, a deletion, and/or a substitution; the substitution is preferably a substitution of conserved amino acids;
optionally, the single-domain antibody comprises a sequence having at least 80%, 85%, 90%, 91%, 92%, 93%, 94%, 95%, 96%, 97%, 98%, 99%, or 100% identity to the sequence set forth in any one of SEQ ID NOs: 6-16.

In some specific embodiments, the single-domain antibody comprises an FR region in the VHH domain set forth in any one of SEQ ID NOs: 6-16;
optionally, the single-domain antibody comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 6-16; the mutation may be selected from an insertion, a deletion, and/or a substitution; the substitution is preferably a substitution of conserved amino acids;
optionally, the single-domain antibody comprises a sequence having at least 80%, 85%, 90%, 91%, 92%, 93%, 94%, 95%, 96%, 97%, 98%, 99%, or 100% identity to the FR region in the VHH domain set forth in any one of SEQ ID NOs: 6-16.

In some specific embodiments, the antibody or the antigen-binding fragment is: (1) a chimeric antibody or a fragment thereof, (2) a humanized antibody or a fragment thereof, or (3) a fully human antibody or a fragment thereof.

In some specific embodiments, the antibody or the antigen-binding fragment comprises or does not comprise an antibody heavy chain constant region; optionally, the antibody heavy chain constant region may be selected from human, Vicugna pacos, mouse, rat, rabbit, and sheep; optionally, the antibody heavy chain constant region may be selected from IgG, IgM, IgA, IgE, and IgD, and the IgG may be selected from IgG1, IgG2, IgG3, and IgG4; optionally, the antibody heavy chain constant region may be selected from an Fc region, a CH3 region, and an intact heavy chain constant region, and preferably, a human Fc region; preferably, the antibody or the antigen-binding fragment is a heavy chain antibody.

In some specific embodiments, the antibody or the antigen-binding fragment is further conjugated to a therapeutic agent or a tracer; preferably, the therapeutic agent is selected from a radioisotope, a chemotherapeutic agent and an immunomodulator, and the tracer is selected from a radiocontrast medium, a paramagnetic ion, a metal, a fluorescent label, a chemiluminescent label, an ultrasound contrast agent, and a photosensitizer; more preferably, the cytotoxic agent is selected from an alkaloid, methotrexate, doxorubicin, a taxane, and a toxin compound; the toxin compound is preferably selected from DM1, DM4, SN-38, MMAE, MMAF, duocarmycin, calicheamicin and DX8951.

In some specific embodiments, the antibody or the antigen-binding fragment specifically binds to human and/or monkey CD5; optionally, the antibody or the antigen-binding fragment binds to human CD5 with a KD greater than 1.00E-6 M, 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M, 1.00E-10 M, 2.00E-10 M, 3.00E-10 M, 4.00E-10 M, 5.00E-10 M, 6.00E-10 M, 7.00E-10 M, 8.00E-10 M, 9.00E-10 M, 1.00E-11 M, or 1.00E-12 M.

In a second aspect, the present disclosure further provides a polypeptide comprising the antibody or the antigen-binding fragment described above, wherein preferably, the polypeptide is further linked to an additional functional molecule; the additional functional molecules may be selected from one or more of a signal peptide, a protein tag or an additional antigen-binding molecule, and a cytokine.

In some specific embodiments, the additional antigen-binding molecule specifically binds to an antigen other than CD5 or binds to a CD5 epitope different from that of the antibody or the antigen-binding fragment described above;
preferably, the antigen other than CD5 may be selected from: CD3, preferably, CD38; CD16, preferably, CD16A; CD19; TGF-beta II receptor; NKG2D; CD40; 4-1BB; CD137 or CD19; EGFR; EGFRvIII; mesothelin; HER2; EphA2; Her3; EpCAM; MUC1; MUC16; CEA; Claudin18.2; folate receptor; Claudin6; WT1; NY-ESO-1; MAGE3; and ASGPR1 or CDH16;
preferably, the additional antigen-binding molecule is an antibody or antigen-binding fragment;
preferably, the polypeptide is a multispecific antigen-binding molecule, for example, bispecific, trispecific, or tetraspecific; more preferably, the multispecific antigen-binding molecule may be divalent, tetravalent, or hexavalent.

In some specific embodiments, the cytokine may be selected from IL2, IL-6, IL-12, IL-15, IL-21, IFN and TNF-alpha.

In a third aspect, the present disclosure further provides a chimeric antigen receptor (CAR); the CAR comprises an extracellular antigen-binding domain, a transmembrane domain, and an intracellular signaling domain; the extracellular antigen-binding domain comprises the antibody or the antigen-binding fragment described above.

In a fourth aspect, the present disclosure further provides an immune effector cell expressing the chimeric antigen receptor described above, or comprising a nucleic acid fragment encoding the chimeric antigen receptor described above, wherein preferably, the immune effector cell is selected from a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell; preferably, the immune effector cell is an autoimmune effector cell or an allogeneic immune effector cell.

In a fifth aspect, the present disclosure further provides an isolated nucleic acid fragment encoding the antibody or the antigen-binding fragment, the polypeptide, or the chimeric antigen receptor described above.

In a sixth aspect, the present disclosure further provides a vector comprising the nucleic acid fragment described above.

In a seventh aspect, the present disclosure further provides a host cell comprising the vector described above, wherein preferably, the cell is a prokaryotic cell or a eukaryotic cell, such as a bacteria (Escherichia coli), a fungus (yeast), an insect cell, or a mammalian cell (a CHO cell or a 293T cell).

In a ninth aspect, the present disclosure further discloses a method for preparing the antibody or the antigen-binding fragment or the polypeptide described above, comprising: culturing the cell described above, and isolating an antibody, an antigen-binding fragment, or a polypeptide expressed by the cell.

In a tenth aspect, the present disclosure further provides a method for preparing the immune effector cell described above, comprising introducing a nucleic acid fragment encoding the CAR described above into the immune effector cell, wherein optionally, the method further comprises initiating expression of the CAR described above in the immune effector cell.

In an eleventh aspect, the present disclosure further provides a pharmaceutical composition comprising the antibody or the antigen-binding fragment, the polypeptide, the immune effector cell, the nucleic acid fragment, or the vector described above, or a product prepared by the method described above; wherein preferably, the pharmaceutical composition further comprises a pharmaceutically acceptable carrier, a diluent, or an adjuvant; and optionally, the pharmaceutical composition further comprises an additional antineoplastic agent.

In a twelfth aspect, the present disclosure further provides a pharmaceutical composition comprising the antibody or the antigen-binding fragment, the polypeptide, the immune effector cell, the nucleic acid fragment, or the vector described above, or a product prepared by the method described above, and a combination of any other active ingredients. “Any other active ingredients” are not defined herein as long as any other active ingredients capable of being combined with the antibody or the antigen-binding fragment, the polypeptide, the immune effector cell, the nucleic acid fragment, or the vector described above, or a product prepared by the method described above to constitute the pharmaceutical composition shall fall within the protection scope of the present disclosure.

In a thirteenth aspect, the present disclosure further provides a method for the treatment of a disease, comprising administering an effective amount of the antibody or the antigen-binding fragment, the polypeptide, the immune effector cell, the nucleic acid fragment, the vector, or the pharmaceutical composition described above, or a product prepared by the method described above to a subject, wherein the disease is preferably selected from a tumor or a cancer and an autoimmune disease; the tumor or the cancer may be selected from a solid tumor and a hematologic tumor; the hematologic tumor may be selected from T-cell lymphoma, chronic lymphoma leukemia, cutaneous T-cell lymphoma, T-cell acute lymphocytic leukemia, and non-Hodgkin's lymphoma; the autoimmune disease may be selected from rheumatoid arthritis and graft-versus-host disease.

In a fourteenth aspect, the present disclosure further provides use of the antibody or the antigen-binding fragment, the polypeptide, the immune effector cell, the nucleic acid fragment, the vector, the host cell, or the pharmaceutical composition described above, or a product prepared by the method described above in preparing a medicament for treating a tumor, a cancer, or an autoimmune disease, wherein preferably, the tumor or the cancer may be selected from a solid tumor and a hematologic tumor; the hematologic tumor may be selected from T-cell lymphoma, chronic lymphoma leukemia, cutaneous T-cell lymphoma, T-cell acute lymphocytic leukemia, and non-Hodgkin's lymphoma; the autoimmune disease may be selected from rheumatoid arthritis and a graft-versus-host disease.

In a fifteenth aspect, the present disclosure further provides use of the antibody or the antigen-binding fragment, the polypeptide, the immune effector cell, the nucleic acid fragment, the vector, or the pharmaceutical composition described above, or a product prepared by the method described above in a medicament for treating a tumor, a cancer, or an autoimmune disease, wherein preferably, the tumor or the cancer may be selected from a solid tumor and a hematologic tumor; the hematologic tumor may be selected from T-cell lymphoma, chronic lymphoma leukemia, cutaneous T-cell lymphoma, T-cell acute lymphocytic leukemia, and non-Hodgkin's lymphoma; the autoimmune disease may be selected from rheumatoid arthritis and a graft-versus-host disease.

In a sixteenth aspect, the present disclosure further provides a kit comprising the antibody or the antigen-binding fragment, the polypeptide, the immune effector cell, the nucleic acid fragment, the vector, the host cell, or the pharmaceutical composition described above, or a product prepared by the method described above.

In a seventeenth aspect, the present disclosure further provides a method for detecting CD5 expression in a biological sample, comprising contacting the biological sample with the antibody or the antigen-binding fragment described above in a condition allowing formation of a complex between the antibody or the antigen-binding fragment described above and CD5, wherein preferably, the method further comprises detecting the formation of the complex, and indicating the presence or an expression level of CD5 in the sample.

In an eighteenth aspect, the present disclosure further provides use of the antibody or the antigen-binding fragment described above in preparing a CD5 assay reagent.

### Definitions of Terms

Unless otherwise defined herein, scientific and technical terms used in correlation with the present disclosure shall have the meanings that are commonly understood by those skilled in the art.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “including”, “comprising”, and “having” herein are used interchangeably and are intended to indicate the inclusion of a solution, implying that there may be elements other than those listed in the solution. Meanwhile, it should be understood that the descriptions “including”, “comprising”, and “having” as used herein also provides the solution of “consisting of . . . ”.

The term “and/or” as used herein includes the meanings of “and”, “or”, and “all or any other combination of elements linked by the term”.

The term “CD5” herein is a kind of cell differentiation antigen and belongs to a cysteine-rich scavenger receptor (SRCR) superfamily, including human CD5 (NCBI: NP_055022.2) and its variants, isoforms and species homologs, where the species homologs include but not limited to monkey CD5 (NCBI: H9ZFB2-1) and its variants and isoforms.

The term “specific binding” herein means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), with lower KD indicating higher affinity. In the case of antibodies, high affinity generally means having a KD of about 10⁻⁶ M or less, about 10⁻⁷ M or less, about 10⁻⁸ M or less, about 1×10⁻⁹ M or less, about 1×10⁻¹⁰ M or less, 1×10⁻¹¹ M or less, or 1×10⁻¹² M or less. KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis.

The term “antigen-binding molecule” herein is used in the broadest sense and refers to a molecule that specifically binds to an antigen. Illustratively, antigen-binding molecules include but are not limited to, antibodies or antibody mimetics. “Antibody mimetic” refers to an organic compound or a binding domain that is capable of specifically binding to an antigen, but is not structurally related to an antibody. Illustratively, antibody mimetics include but are not limited to, affibody, affitin, affilin, a designed ankyrin repeat protein (DARPin), a nucleic acid aptamer, or a Kunitz domain peptide.

The term “antibody” herein is used in the broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, e.g., stabilize the three-dimensional structure of the antibody, and fully synthetic scaffolds comprising, e.g., biocompatible polymers. See, e.g., Korndorfer et al., 2003, Proteins: Structure, Function, and Bioinformatics, 53 (1): 121-129 (2003); and Roque et al., Biotechnol. Prog. 20:639-654 (2004). Such scaffolds may also include non-antibody derived scaffolds, such as scaffold proteins known in the art to be useful for grafting CDRs, including but not limited to tenascin, fibronectin, peptide aptamers, and the like.

The term “antibody” herein includes a typical “four-chain antibody”, which is an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, a heavy chain constant region CH3 domain; moreover, when the full-length antibody is of IgE isoform, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are connected to each other and to the light chains through disulfide bonds to form a Y-shaped structure. The heavy chain constant regions of an immunoglobulin differ in their amino acid composition and arrangement, and thus in their antigenicity. Accordingly, “immunoglobulin” herein can be divided into five classes, or called isoforms of the immunoglobulin, namely IgM, IgD, IgG, IgA and IgE, with their corresponding heavy chains being μ, δ, γ, α, and ε chains, respectively. The Ig of the same class can be divided into different subclasses according to the differences in the amino acid composition of the hinge regions and the number and location of disulfide bonds in the heavy chains. For example, IgG can be divided into IgG1, IgG2, IgG3, and IgG4; and IgA can be divided into IgA1 and IgA2. Light chains are divided into κ or λ chains according to differences in the constant regions. Each of the five classes of Ig may have a κ chain or a λ chain.

The term “antibody” herein also includes antibodies exclusive of a light chain, e.g., heavy-chain antibodies (HCAbs) produced by Camelus dromedarius, Camelus bactrianus, Lama glama, Lama guanicoe, Vicugna pacos, and the like, as well as immunoglobulin new antigen receptors (IgNAR) found in Chondrichthyes, e.g., shark.

As used herein, the term “heavy chain antibody” refers to an antibody lacking a light chain of a conventional antibody. The term specifically includes but not limited to homodimeric antibodies containing a VH antigen-binding domain, CH2 and CH3 constant domains in absence of a CH1 domain.

The terms “VHH domain”, “nanobody”, and “single-domain antibody (sdAb)” herein have the same meaning and can be used interchangeably, and refer to a single-domain antibody consisting of only one heavy chain variable region constructed by cloning a variable region of a heavy chain antibody, which is the smallest antigen-binding fragment having the full function. Generally, a single-domain antibody consisting of only one heavy chain variable region is constructed by obtaining a heavy chain antibody naturally lacking a light chain and a heavy chain constant region 1 (CH1) and then cloning a variable region of an antibody heavy chain.

The “heavy chain antibody” and “single-domain antibody”, “VHH domain” and “nanobody” will be further described by reference to: Hamers-Casterman, et al., Nature. 1993; 363; 446-8; Muyldermans' review article (Reviews in Molecular Biotechnology 74:277-302, 2001); and the following patent applications which are mentioned as general background art: WO 94/04678, WO 95/04079 and WO 96/34103; WO94/25591, WO 99/37681, WO 00/40968, WO 00/43507, WO 00/65057, WO 01/40310, WO 01/44301, EP 1134231 and WO 02/48193; WO97/49805, WO 01/21817, WO 03/035694, WO 03/054016 and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787 and WO 06/122825 as well as other prior arts mentioned in these applications.

“Antibody” herein may be derived from any animal, including but not limited to humans and non-human animals; the non-human animals may be selected from primates, mammals, rodents, and vertebrates, such as Camelidae species, Lama glama, Lama guanicoe, Vicugna pacos, sheep, rabbit, mouse, rat, or Chondrichthyes (e.g., shark).

The term “multispecific” means having at least two antigen-binding sites, each of which binds to a different epitope of the same antigen or a different epitope of a different antigen. Thus, terms such as “bispecific”, “trispecific”, and “tetraspecific” refer to the number of different epitopes to which an antibody/antigen-binding molecule can bind.

The term “valency” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

“Full-length antibody”, “complete antibody”, and “intact antibody” herein are used interchangeably and refer to an antibody having a substantially similar structure to a natural antibody.

“Antigen-binding fragment” and “antibody fragment” herein are used interchangeably and do not have the entire structure of an intact antibody, but comprise only a partial or partial variant of the intact antibody that has the ability to bind to an antigen. Illustratively, “antigen-binding fragment” or “antibody fragment” herein includes but not limited to, a Fab, an F(ab′)₂, a Fab′, a Fab′-SH, an Fd, an Fv, an scFv, a diabody, and a single-domain antibody.

“Chimeric antibody” herein refers to the following antibody which has a variable sequence of immunoglobulin derived from a microbial source (e.g., rats, mice, rabbits or Vicugna pacos) and a constant region of immunoglobulin derived from different organisms (e.g., human). A method for producing the chimeric antibody is known in the art. See, for example, Morrison, 1985, Science 229 (4719): 1202-7; Oi, et al., 1986, Bio Techniques 4:214-221; Gillies, et al., 1985 J Immunol Methods 125:191-202; the content of which is incorporated herein by reference.

The term “humanized antibody” herein refers to a genetically engineered non-human antibody that has an amino acid sequence modified to increase homology to the sequence of a human antibody. Generally, all or part of the CDR regions of a humanized antibody are derived from a non-human antibody (donor antibody), and all or part of the non-CDR regions (e.g., variable region FRs and/or constant regions) are derived from a humanized immunoglobulin (receptor antibody). The humanized antibody generally retains or partially retains the desired properties of the donor antibody, including but not limited to, antigen specificity, affinity, reactivity, the ability to increase the activity of immune cells, the ability to enhance an immune response, and the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “variable region” herein refers to a region of the heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conserved framework regions (FRs) and three hypervariable regions (HVRs). See, e.g., Kindt et al., Kuby Immunology, 6th ed., W. H. Freeman and Co., p. 91 (2007). A single VH or VL domain can be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it can form precise complementarity to an epitope in a spatial structure, wherein the heavy chain variable chain CDR may be abbreviated as HCDR and the light chain variable chain CDR may be abbreviated as LCDR. The terms “framework region” or “FR region” are used interchangeably and refer to those amino acid residues of an antibody heavy chain variable region or light chain variable region, other than the CDRs. Generally, a typical antibody variable region consists of 4 FR regions and 3 CDR regions in the following order: FR1-CDR1-FR2-CDR2-FR3-CDR3-FR4.

For further description of the CDRs, reference is made to Kabat et al., J. Biol. Chem., 252:6609-6616 (1977); Kabat et al., U.S. department of health and public services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Plückthun, J. Mol. Biol., 309:657-670 (2001). “CDR” herein may be labeled and defined in a way well known in the art, including but not limited to, the Kabat numbering scheme, the Chothia numbering scheme, or the IMGT numbering scheme; the tool sites used include but not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196:901-917; Chothia et al., (1989) Nature 342:878-883). The term “IMGT numbering scheme” herein generally refers to a numbering scheme based on the international ImMunoGeneTics information system (IMGT) initiated by Lefranc et al., see Lefranc et al., Dev. Comparat. Immunol. 27:55-77, 2003.

The term “heavy chain constant region” herein refers to the carboxyl-terminal portion of an antibody heavy chain that is not directly involved in the binding of the antibody to an antigen, but exhibits effector functions, such as interaction with an Fc receptor, which has a more conserved amino acid sequence relative to the variable domain of the antibody. The “heavy chain constant region” may be selected from: a CH1 domain, a hinge region, a CH2 domain, a CH3 domain, and a variant or fragment thereof. The “heavy chain constant region” includes a “full-length heavy chain constant region” having a structure substantially similar to that of a natural antibody constant region, and a “heavy chain constant region fragment” including only a portion of the full-length heavy chain constant region. Illustratively, a typical “full-length antibody heavy chain constant region” consists of the CH1 domain-hinge region-CH2 domain-CH3 domain. When the antibody is IgE, it further comprises a CH4 domain; and when the antibody is a heavy chain antibody, it does not include a CH1 domain. Illustratively, a typical “heavy chain constant region fragment” may be selected from Fc and CH3 domains.

The term “light chain constant region” herein refers to the carboxyl-terminal portion of an antibody light chain that is not directly involved in the binding of the antibody to an antigen. The light chain constant region may be selected from a constant κ domain and a constant λ domain.

The term “Fc region” herein is used to define the C-terminus region of an antibody heavy chain containing at least a portion of the constant region. The “Fc region” includes Fc regions of native sequences and variant Fc regions. Illustratively, the Fc region in a human IgG heavy chain may extend from Cys226 or Pro230 to the carboxyl-terminus of the heavy chain. However, the antibody generated from a host cell may be translated and then cut; one or more, in particular one or two amino acids are cut off from the C-terminus of the heavy chain thereof. Therefore, the antibody generated by a host cell by the expression of a specific nucleic acid molecule encoding a full-length heavy chain may include a full-length heavy chain, or may include cutting variants of the full-length heavy chain. It may be such a situation when the final two C-terminal amino acids of the heavy chain are glycine (G446) and lysine (K447, the numbering way is in accordance with the Kabat EU index). Therefore, the C-terminal lysine (Lys447), or the C-terminal glycine (Gly446) and lysine (Lys447) of the Fc region may or may not be present.

Typically, the IgG Fc region comprises IgG CH2 and IgG CH3 domains; optionally, the IgG Fc region may further comprise a complete or partial hinge region, but exclude a CH1 domain. The “CH2 domain” in the human IgG Fc region generally extends from amino acid residues at the site of about 231 to amino acid residues at the site of about 340. In one embodiment, a carbohydrate chain is attached to the CH2 domain. The CH2 domain herein may be a CH2 domain of a native sequence or a variant CH2 domain. “CH3 domain” comprises the residues at the C-terminus of the CH2 domain in the Fc region (namely, from amino acid residues of IgG at the site of about 341 to amino acid residues at the site of about 447). The CH3 domain herein may be a CH3 domain of a native sequence or a variant CH3 domain (for example, the CH3 domain having a “bulge” (“knob”) introduced into one chain thereof and a “cavity” (“hole”) accordingly introduced into another chain thereof; see U.S. Pat. No. 5,821,333, the content of which is definitely incorporated herein by reference). As described herein, such kind of variant CH3 domain may be used to promote the heterodimerization of two different antibody heavy chains.

Unless otherwise specified herein, the numbering of amino acid residues in the Fc region or constant region conforms to the EU numbering scheme, also known as the EU index, as described in Kabat et al., Sequences of Proteins of Immunological Interest, 5th Ed., Public Health Service, National Institutes of Health, Bethesda, MD, 1991.

The term “conserved amino acid” herein generally refers to amino acids that belong to the same class or have similar characteristics (e.g., charge, side chain size, hydrophobicity, hydrophilicity, backbone conformation, and rigidity).

Illustratively, the following six groups are examples of amino acids that are considered to be conserved replacement of each other:
1) alanine (A), serine(S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48:444-453; available at www.gcg.com) which has been integrated into the GAP program of the GCG software package, using the Blossom 62 matrix or PAM250 matrix and gap weight of 16, 14, 12, 10, 8, 6, or 4 and length weight of 1, 2, 3, 4, 5, or 6. For another example, the percent identity between two nucleotide acid sequences is determined with the GAP program of the GCG software package (available at www.gcg.com), using the NWSgapdna.CMP matrix and gap weight of 40, 50, 60, 70 or 80 and length weight of 1, 2, 3, 4, 5 or 6. A particularly preferred parameter set (and one that should be used unless otherwise stated) is a Blossom 62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4:11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or correlated sequences. For example, such searches can be performed using the NBLAST and XBLAST programs (version 2.0) of Altschul et al., (1990) J. Mol. Biol., 215:403-10. BLAST nucleotide searches can be performed with the NBLAST program, with a score of 100 and a word length of 12, to obtain nucleotide sequences homologous to the nucleic acid molecule of the present disclosure. BLAST protein searches can be performed using the XBLAST program, with a score of 50 and a word length of 3, to obtain amino acid sequences homologous to the protein molecule of the present disclosure. To obtain gapped alignment results for the purpose of comparison, gapped BLAST can be used as described in Altschul et al. (1997) Nucleic Acids Res. 25:3389-3402. When using the BLAST and gapped BLAST programs, the default parameters of the respective programs (e.g., XBLAST and NBLAST) can be used. See www.ncbi.nlm.nih.gov.

The term “chimeric antigen receptor (CAR)” herein refers to an artificial cell surface receptor engineered to be expressed on an immune effector cell and specifically binded to an antigen, which comprises at least (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. In this context, the term “nucleic acid molecule” encompasses deoxyribonucleic acid (DNA), including, e.g., complementary DNA (cDNA) and genomic DNA; ribonucleic acid (RNA), in particular in the synthetic form of messenger RNA (mRNA), DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as a vector for direct expression of the antibodies of the present disclosure in vitro and/or in vivo, e.g., in a host or a patient. Such DNA (e.g., cDNA) or RNA (e.g., mRNA) vectors may be unmodified or modified. For example, mRNA can be chemically modified to enhance the stability of the RNA vector and/or the expression of the encoded molecule such that the mRNA can be injected into a subject to produce antibodies in vivo (see, e.g., Stadler et al., Nature Medicine 2017, published online, Jun. 12, 2017, doi: 10.1038/nm.4356 or EP 2 101 823 B1). An “isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location.

The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered.

The term “treatment” herein refers to surgical or therapeutic treatment for the purpose of preventing, slowing (reducing) the progression of an undesired physiological or pathological change, e.g., cancer or tumor. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (whether partial or complete), whether detectable or undetectable. Subjects in need of treatment include subjects already suffering from a disorder or disease as well as subjects susceptible to a disorder or disease or subjects for whom prevention of a disorder or disease is intended. When referring to terms such as slow, moderate, reduce, ameliorate, and alleviate, their meanings also include elimination, disappearance, nonoccurrence, etc.

The term “subject” herein refers to an organism that receives treatment for a particular disease or disorder described herein. Illustratively, the “subject” includes mammals, such as humans, primates (e.g., monkey), or non-primate mammals, that are being treated for a disease or disorder.

The term “effective amount” herein refers to an amount of a therapeutic agent that is effective to prevent or alleviate symptoms of a disease or the progression of the disease when administered to a cell, tissue or subject alone or in combination with another therapeutic agent. “Effective amount” also refers to an amount of a compound that is sufficient to alleviate symptoms, e.g., to treat, cure, prevent, or alleviate the associated medical disorder, or to increase the rate at which such disorder is treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

The term “EC₅₀” herein refers to the half maximum effective concentration, which includes the antibody concentration that induces a halfway response between the baseline and maximum after a specified exposure time. EC₅₀ essentially represents the antibody concentration by which 50% of its maximum effect is observed, and can be measured by methods known in the art.

### BRIEF DESCRIPTION OF THE DRAWING

FIG. 1A shows assay results for serum antibody titer of Vicugna pacos after being immunized by a human CD5 protein by ELISA;

FIG. 1B shows assay results for serum antibody titer of Vicugna pacos after being immunized by a human CD5 protein by FACS;

FIG. 2 shows assay results for binding reactions of control antibodies with a human CD5-his protein by ELISA;

FIG. 3 shows assay results for binding reactions of control antibodies with Jurkat cells by FACS;

FIG. 4 shows assay results for Jurkat cells of an endogenous cell line by FACS;

FIGS. 5A-5D show assay results for screening for CHO-K1 cells plasmid-transfected with a human CD5 by FACS;

FIG. 6 shows assay results for screening for CHO-K1 cells plasmid-transfected with a monkey CD5 by FACS;

FIGS. 7A-7B show assay results for binding reactions of VHH-hFc with a human CD5-his protein by ELISA;

FIGS. 8A-8B show assay results for binding reactions of VHH-hFc with Jurkat cells by FACS;

FIGS. 8C-8D show assay results for binding reactions of VHH-hFc with CHO-K1-human CD5 cells by FACS;

FIG. 9A shows assay results for binding reactions of 1 nM and 10 nM VHH-hFc antibodies with Jurkat and Raji cells by FACS;

FIG. 9B shows assay results for binding reactions of 1 nM and 10 nM VHH-hFc antibodies with CHO-K1 cells and CHO-K1-human CD5 by FACS;

FIG. 10 shows assay results for binding reactions of VHH-hFc with a murine CD5-his protein by ELISA;

FIG. 11 shows assay results for binding reactions of VHH-hFc with HEK293T-monkey CD5 by FACS;

FIGS. 12A-12B show assay results for affinity of VHH-hFc with a human CD5 protein by SPR;

FIGS. 13A-13B show an inhibition ratio between VHH-hFc antibodies detected by competitive ELISA; and

FIG. 14 shows classification of epitopes of VHH-hFc.

### DETAILED DESCRIPTION

The present disclosure is further described below with reference to specific examples; the advantages and features of the present disclosure will become more apparent with the description. Experimental procedures without specified conditions in the examples are conducted according to conventional conditions or conditions recommended by the manufacturer. Reagents or instruments without specified manufacturers used herein are conventional products that are commercially available.

The examples herein are exemplary only, and do not limit the scope of the present disclosure in any way. It will be understood by those skilled in the art that various modifications or substitutions may be made to the technical solutions of the present disclosure in form and details without departing from the spirit and scope of the present disclosure, and that these modifications and substitutions shall fall within the protection scope of the present disclosure.

All the sequences provided in the present disclosure are specifically referring to the description and Sequence Listing. If the sequences disclosed in the description are different from the sequences in the Sequence Listing, please be subject to the description.

### Example 1. Screening of Heavy Chain Single-Domain Antibodies Against CD5

1.1 Vicugna pacos Immunization and Serum Titer Assay

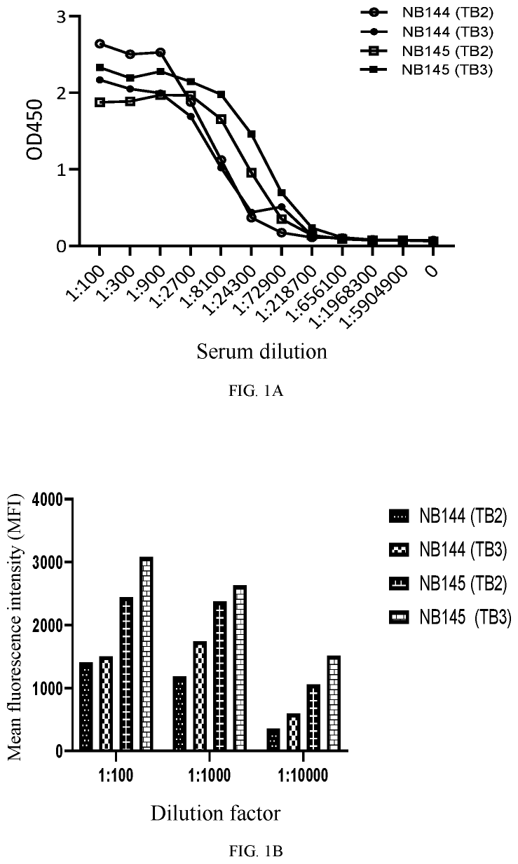

The human CD5 (Arg25-Pro372)-His protein for immunization was purchased from Acro (Cat. No. CD5-H52H5). Two Vicugna pacos (Alpaca, No. NB144 and No. NB145) were chosen and immunized for four times, separately, and the immunization was performed every other 3 weeks each time; peripheral blood was collected and subjected to serum isolation after the third and fourth immunizations, and then antibody titer and specificity against human CD5 in the serum were assayed by ELISA and FACS. The results are shown in FIGS. 1A-1B and Table 1. Table 1 indicates that serum from the Vicugna pacos immunized by the human CD5 protein presented antigen-antibody reactions with a maximum dilution of around 210,000 (1:218700). The blank control is 1% (w/w) BSA, and the serum sample refers to serum from the Vicugna pacos on the 7th day after the third (TB2) and fourth (TB3) immunizations; data in the table are values of OD_{450 nm}.

TABLE 1
Titer of serum from the Vicugna pacos after being immunized
with a human CD5 protein assayed by ELISA
OD450 nm

| | Batch | | | |
|---|---|---|---|---|
| Serum dilution | NB144(TB2) | NB144(TB3) | NB145(TB2) | NB145(TB3) |
| 1:100 | 2.64 | 2.17 | 1.88 | 2.33 |
| 1:300 | 2.51 | 2.05 | 1.89 | 2.19 |
| 1:900 | 2.53 | 2.00 | 1.97 | 2.28 |
| 1:2700 | 1.88 | 1.69 | 1.97 | 2.15 |
| 1:8100 | 1.12 | 1.02 | 1.66 | 1.98 |
| 1:24300 | 0.37 | 0.44 | 0.96 | 1.46 |
| 1:72900 | 0.17 | 0.51 | 0.35 | 0.70 |
| 1:218700 | 0.11 | 0.14 | 0.14 | 0.24 |
| 1:656100 | 0.10 | 0.10 | 0.09 | 0.11 |
| 1:1968300 | 0.08 | 0.07 | 0.07 | 0.08 |
| 1:5904900 | 0.08 | 0.07 | 0.07 | 0.07 |
| Blank control | 0.07 | 0.07 | 0.06 | 0.07 |

### 1.2 Library Construction

A total of 100 mL peripheral blood was collected from Vicugna pacos after the third and fourth immunizations, and PBMC was isolated with a lymphocyte separation medium; total RNA was extracted with an RNAiso Plus reagent; the extracted RNA was reversely transcribed into cDNA with a PrimeScript™ II 1st Strand cDNA Synthesis Kit (Takara, Cat. No. 6210A). Nested PCR was applied to amplify the nucleic acid fragment encoding a heavy chain antibody variable region:

The first round of PCRs:

Forward primer:
(SEQ ID NO: 1)
CTTGGTGGTCCTGGCTGC;
Reverse primer:
(SEQ ID NO: 2)
GGTACGTGCTGTTGAACTGTTCC.

The second round of PCRs: products from the first round of PCRs were used as templates.

Forward primer:
(SEQ ID NO: 3)
CATGCCATGACTGTGGCCCAGGCGGCCCAGKTGCAGCTCGTGGAGTC;
Reverse primer-1:
(SEQ ID NO: 4)
CATGCCATGACTCGCGGCCGGCCTGGCCATGGGGGTCTTCGCTGTGGTG
CG;
Reverse primer-2:
(SEQ ID NO: 5)
CATGCCATGACTCGCGGCCGGCCTGGCCGTCTTGTGGTTTTGGTGTCTT
GGG

The nucleic acid fragment of the target single-domain antibody was recovered and then cloned into a vector pcomb3XSS used for phage display using a restriction endonuclease SfiI. The product was then electrotransformed into E. coli electroporation competent cells TG1, and a single-domain antibody phage display library against CD5 was constructed and assayed. The size of the reservoir was calculated to be 5.0×10⁹ by gradient dilution plating. To determine the insertion rate of the library, 48 clones were randomly selected and subjected to colony PCR. The results showed that the insertion rate is up to 100%.

### 1.3 Panning of the Single-Domain Antibody Against CD5

A plate was coated with human CD5-His protein (Acro, Cat. No. CD5-H52H5) with 0.5 μg/well, and placed over the night at 4° C.; the plate was blocked with 3% BSA-PBS for 1 h at 37° C. in the next day, and then added with 100 μl phage display library for incubation for 1 h at 37° C., afterwards, the plate was washed for 6 times with PBST and washed twice with PBS, so as to wash out unbound phages. Finally, 100 μL Gly-HCl eluent was added to elute the phage specifically binding to CD5, thus enriching positive clones.

### 1.4 Screening of Specific Single Positive Clones Using Phage Enzyme-Linked Immunosorbent Assay

The CD5-binding positive phages obtained after panning were infected with blank E. coli and plated. 96 single colonies were then selected, amplified and cultured, separately. The plates were coated with the human CD5-His protein, separately, at 4° C. over the night, and added with the phage cultural supernatant, then incubated for 1 h at 37° C. A TMB color-developing solution was added for color developing after washing the plates, and optical density was measured at a wavelength of 450 nm. CD5-His positive clones were selected and sequenced. The sequencing results were analyzed with MOE software and an evolutionary tree was constructed according to the amino acid sequences of the VHH encoded protein; after the sequences close to each other on the evolutionary tree were eliminated according to sequence similarity, the following VHH antibodies (see details in Table 2) were selected to analyze the CDR regions thereof by a bioinformatics method, and subjected to the subsequent VHH-hFc production and identification (detailed results are specifically as shown in Table 3). The analysis modes include: websites http://www.imgt.org/3Dstructure-DB/cgi/DomainGapAlign.cgi#results (IMGT); http://www.abysis.org/abysis/sequence_input/key_annotation/key_annotation.cgi (IMGT, Kabat and Chothia); http://cao.labshare.cn/AbRSA/abrsa.php (Kabat and Chothia).

TABLE 2
Sequence information of VHH antibodies

| Antibody name | |
|---|---|
| and No. | Sequence information |
| S005 NB 144-3 | QLQLVESGGGLVQPGGSLRLSCAASGFTFSSYAMSWYRQAPGKERELVAAIT |
| (SEQ ID NO: 6) | SAGGSTTYADSVKDRFTISRDNAKNTVSLQMNSLKPEDTAVYYCNAYCTGY |
| | VCHEGGYDYWGQGTQVTVSS |
| S005 NB144-6 | QVQLVESGGGLVQAGGSLRLSCAASGSIFSISVMGWYRQAPGKERETVAVIS |
| (SEQ ID NO: 7) | SGGSTYYADSVKGRFTISRDNAKNTLYLQMNSLKPEDTAVYYCNADFETAS |
| | GSPLYESWGQGTQVTVSS |
| S005 NB 144-7 | EVQVVESGGGLVQAGGSLRLSCAASGSIFSINLLGWYRQAPGKERESVAILS |
| (SEQ ID NO: 8) | SGGSTYYADSVKGRFTISRDNVKNMLYLQMNSLKPEDTAVYYCNAVGGIAT |
| | LRDYWGQGTQVTVSS |
| S005 NB144-31 | EVQVVESGGGLVQPGGSLRLSCAASGFTLDYYAIGWFRQAPGKEREGLSCIS |
| (SEQ ID NO: 9) | TSGGSTNYADSVKGRFTISRDNAKNTVYLQMNSLKPEDTAVYYCAASSFWD |
| | CAVVGTGSPKTGHLESVFGSWGQGTQVTVSS |
| S005 NB 144-43 | QVQLVESGGGLVQPGGSLRLSCAASGFTWDYYAIGWFRQAPGKEREGVSCI |
| (SEQ ID NO: 10) | DSSGGSINYLDSVKGRFTVSRDNAKNTVYLQMDSLKPEDTAIYYCAASSYW |
| | DCAVPDTGSPKTGHLESYFGSWGQGTQVIVSS |
| S005 NB 144-73 | EVQVVESGGGLVQAGGSLRLSCAASGDTICISAMYWYRQAPGKAREMVAA |
| (SEQ ID NO: 11) | ITSGGSTYYEDSVMGRFTIFRENAKNTVYLQMNSLKPEDTAVYYCNADIAG |
| | HNCSGYLKEYWGQGTQVTVSA |
| S005 NB 145-3 | QVQLVESGGGLVQPGGSLRLSCAASGFSLDYYAIGWFRQAPGKEREGVSYIS |
| (SEQ ID NO: 12) | PSDGRTYYADSVKGRFTISRDNAKNTVYLQMNSLKPEDTAVYYCATDWGST |
| | SFDYGLKLSPTQYGYWGQGTQVTVSS |
| S005 NB145-22 | QVQLVESGGGLVQAGGSLRLSCAVSGRTLSTVVVGWFRQTPGREREFVAVS |
| (SEQ ID NO: 13) | RSGGATGYADSVKDRFTISRDNAKNTVALQMNSLKPEDTAVYYCAGRGSGL |
| | YGSSWYNQIQEYDGWGQGTQVTVSS |
| S005 NB 145-74 | EVqLVESGGGLVQAGGSLRLSCAASESTFSITAMGWYRQAPGKQRELVAAIT |
| (SEQ ID NO: 14) | SPGGSTNYADSVKGRFTISRDNAKNTMLLQMNSLKPEDTAVYYCNADINHG |
| | SGYYTVSWGQGTQVTVSS |
| S005 NB145-92 | EVqLVESGGGLVQPGGSLRLSCAASGFSLDYYAIGWFRQAPGKEREGVSYIS |
| (SEQ ID NO: 15) | ASDGSTYYADSVKGRFTISRDNAKNTVYLQMNSLKPEDTAVYYCATDWGS |
| | GSFNYGLRLSPTQYDYWGQGTQVTVSS |
| S005 NB 144-4 | EVQVVESGGGLVQAGGSLRLSCAASGSIFSINAMGWYRQAPGKEREVVAAI |
| (SEQ ID NO: 16) | SNGGNTYYEDSVMGRFTISRDNAKGTLYLQMNGLKPEDTAVYYCNADTVV |
| | GGGLLTEVWGQGTLVTVSS |

TABLE 3
Information about CDR regions of VHH antibodies

| Antibody | Analysis | | | |
|---|---|---|---|---|
| name | mode | CDR1 | CDR2 | CDR3 |
| S005 | IMGT | GFTFSSYA | ITSAGGST | NAYCTGYVCHEGGYDY |
| NB144-3 | | (SEQ ID NO: 17) | (SEQ ID NO: 18) | (SEQ ID NO: 19) |
| | Kabat | SYAMS | AITSAGGSTTYADSVKD | YCTGYVCHEGGYDY |
| | | (SEQ ID NO: 20) | (SEQ ID NO: 21) | (SEQ ID NO: 22) |
| | Chothia | GFTFSSY | TSAGGS | YCTGYVCHEGGYDY |
| | | (SEQ ID NO: 23) | (SEQ ID NO: 24) | (SEQ ID NO: 25) |
| S005 | IMGT | GSIFSISV | ISSGGST | NADFETASGSPLYES |
| NB144-6 | | (SEQ ID NO: 26) | (SEQ ID NO: 27) | (SEQ ID NO: 28) |
| | Kabat | ISVMG | VISSGGSTYYADSVKG | DFETASGSPLYES |
| | | (SEQ ID NO: 29) | (SEQ ID NO: 30) | (SEQ ID NO: 31) |
| | Chothia | GSIFSIS | SSGGS | DFETASGSPLYES |
| | | (SEQ ID NO: 32) | (SEQ ID NO: 33) | (SEQ ID NO: 34) |
| S005 | IMGT | GSIFSINL | LSSGGST | NAVGGIATLRDY |
| NB144-7 | | (SEQ ID NO: 35) | (SEQ ID NO: 36) | (SEQ ID NO: 37) |
| | Kabat | INLLG | ILSSGGSTYYADSVKG | VGGIATLRDY |
| | | (SEQ ID NO: 38) | (SEQ ID NO: 39) | (SEQ ID NO: 40) |
| | Chothia | GSIFSIN | SSGGS | VGGIATLRDY |
| | | (SEQ ID NO: 41) | (SEQ ID NO: 42) | (SEQ ID NO: 43) |
| S005 | IMGT | GFTLDYYA | ISTSGGST | AASSFWDCAVVGTGSPKTGHLESVFGS |
| NB144-31 | | (SEQ ID NO: 44) | (SEQ ID NO: 45) | (SEQ ID NO: 46) |
| | Kabat | YYAIG | CISTSGGSTNYADSVKG | SSFWDCAVVGTGSPKTGHLESVFGS |
| | | (SEQ ID NO: 47) | (SEQ ID NO: 48) | (SEQ ID NO: 49) |
| | Chothia | GFTLDYY | STSGGS | SSFWDCAVVGTGSPKTGHLESVFGS |
| | | (SEQ ID NO: 50) | (SEQ ID NO: 51) | (SEQ ID NO: 52) |
| S005 | IMGT | GFTWDYYA | IDSSGGSI | AASSYWDCAVPDTGSPKTGHLESYFGS |
| NB144-43 | | (SEQ ID NO: 53) | (SEQ ID NO: 54) | (SEQ ID NO: 55) |
| | Kabat | YYAIG | CIDSSGGSINYLDSVKG | SSYWDCAVPDTGSPKTGHLESYFGS |
| | | (SEQ ID NO: 56) | (SEQ ID NO: 57) | (SEQ ID NO: 58) |
| | Chothia | GFTWDYY | DSSGGS | SSYWDCAVPDTGSPKTGHLESYFGS |
| | | (SEQ ID NO: 59) | (SEQ ID NO: 60) | (SEQ ID NO: 61) |
| S005 | IMGT | GDTICISA | ITSGGST | NADIAGHNCSGYLKEY |
| NB144-73 | | (SEQ ID NO: 62) | (SEQ ID NO: 63) | (SEQ ID NO: 64) |
| | Kabat | ISAMY | AITSGGSTYYEDSVMG | DIAGHNCSGYLKEY |
| | | (SEQ ID NO: 65) | (SEQ ID NO: 66) | (SEQ ID NO: 67) |
| | Chothia | GDTICIS | TSGGS | DIAGHNCSGYLKEY |
| | | (SEQ ID NO: 68) | (SEQ ID NO: 69) | (SEQ ID NO: 70) |
| S005 | IMGT | GFSLDYYA | ISPSDGRT | ATDWGSTSFDYGLKLSPTQYGY |
| NB145-3 | | (SEQ ID NO: 71) | (SEQ ID NO: 72) | (SEQ ID NO: 73) |
| | Kabat | YYAIG | YISPSDGRTYYADSVKG | DWGSTSFDYGLKLSPTQYGY |
| | | (SEQ ID NO: 74) | (SEQ ID NO: 75) | (SEQ ID NO: 76) |
| | Chothia | GFSLDYY | SPSDGR | DWGSTSFDYGLKLSPTQYGY |
| | | (SEQ ID NO: 77) | (SEQ ID NO: 78) | (SEQ ID NO: 79) |
| S005 | IMGT | GRTLSTVV | VSRSGGAT | AGRGSGLYGSSWYNQIQEYDG |
| NB145-22 | | (SEQ ID NO: 80) | (SEQ ID NO: 81) | (SEQ ID NO: 82) |
| | Kabat | TVVVG | VSRSGGATGYADSVKD | RGSGLYGSSWYNQIQEYDG |
| | | (SEQ ID NO: 83) | (SEQ ID NO: 84) | (SEQ ID NO: 85) |
| | Chothia | GRTLSTV | RSGGA | RGSGLYGSSWYNQIQEYDG |
| | | (SEQ ID NO: 86) | (SEQ ID NO: 87) | (SEQ ID NO: 88) |
| S005 | IMGT | ESTFSITA | ITSPGGST | NADINHGSGYYTVS |
| NB145-74 | | (SEQ ID NO: 89) | (SEQ ID NO: 90) | (SEQ ID NO: 91) |
| | Kabat | ITAMG | AITSPGGSTNYADSVKG | DINHGSGYYTVS |
| | | (SEQ ID NO: 92) | (SEQ ID NO: 93) | (SEQ ID NO: 94) |
| | Chothia | ESTESIT | TSPGGS | DINHGSGYYTVS |
| | | (SEQ ID NO: 95) | (SEQ ID NO: 96) | (SEQ ID NO: 97) |
| S005 | IMGT | GFSLDYYA | ISASDGST | ATDWGSGSFNYGLRLSPTQYDY |
| NB145-92 | | (SEQ ID NO: 98) | (SEQ ID NO: 99) | (SEQ ID NO: 100) |
| | Kabat | YYAIG | YISASDGSTYYADSVKG | DWGSGSFNYGLRLSPTQYDY |
| | | (SEQ ID NO: 101) | (SEQ ID NO: 102) | (SEQ ID NO: 103) |
| | Chothia | GFSLDYY | SASDGS | DWGSGSFNYGLRLSPTQYDY |
| | | (SEQ ID NO: 104) | (SEQ ID NO: 105) | (SEQ ID NO: 106) |
| S005 | IMGT | GSIFSINA | ISNGGNT | NADTVVGGGLLTEV |
| NB144-4 | | (SEQ ID NO: 107) | (SEQ ID NO: 108) | (SEQ ID NO: 109) |
| | Kabat | INAMG | AISNGGNTYYEDSVMG | DTVVGGGLLTEV |
| | | (SEQ ID NO: 110) | (SEQ ID NO: 111) | (SEQ ID NO: 112) |
| | Chothia | GSIFSIN | SNGGN | DTVVGGGLLTEV |
| | | (SEQ ID NO: 113) | (SEQ ID NO: 114) | (SEQ ID NO: 115) |

### Example 2. Preparation of VHH-hFc, Control Antibodies and Polyclonal Antibody Serum

### 2.1 Expression and Purification of VHH-hFc

Sequences of the VHH variable region were recombined into an expression vector BI3.4-huIgG1 containing a signal peptide and human IgG1 Fc (SEQ ID NO: 116); plasmids were prepared according to established standard molecular biology methods. See Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from Suzhou Yiyan Biotech Co., Ltd.) were transiently transfected with the expression vector according to PEI (purchased from Polysciences, Cat. No. 24765-1) instructions, cultured at 37° C. for 5 consecutive days using FreeStyle™ 293 (Thermofisher scientific, Cat. No. 12338018), and centrifuged to remove cell components, to obtain culture supernatants containing the VHH-hFc. The culture supernatants were each loaded on a Protein A chromatography column (the Protein A packing, AT Protein A Diamond and the column BXK16/26 were purchased from Bestchrom), washed with a PBS phosphate buffer (pH 7.4), then washed with 20 mM PB, 1 M NaCl, pH 7.2, and finally subjected to elution with a citrate buffer at pH 3.4. An hFc-tagged antibody eluted from the Protein A chromatography column was collected, neutralized with 1/10 volumes of 1 M Tris (pH 8.0), and dialyzed with PBS at 4° C. over the night, and the dialyzed protein was subjected to sterile filtration through a 0.22 μM filter membrane, subpackaged, and stored at −80° C.

Sequence of human IgG1 Fc (SEQ ID NO: 116): EPKSADKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSN KALPAPIEKTISKAKGQPREPQVYTLPPSREEMTKNQVSLTCLVKGFYPSDIAVEWE SNGQPENNYKTTPPVLDSDGSFFLYSKLTVDKSRWQQGNVFSCSVMHEALHNHYT QKSLSLSPGK

### 2.2 Preparation and Identification of the Control Antibodies

H65 is an antibody to identify human CD5; the heavy chain variable region and light chain variable region of H65 were obtained according to the U.S. Pat. No. 5,621,083. The VH and VL of the H65 antibody and human IgG1 Fc were linked in the order from N-terminus to C-terminus; VH and VL were linked through 3 GGGGS connexons to form a scFv-hFc form; corresponding nucleotide sequences thereof were separately cloned onto a pTT5 vector (completed by GENERAL BIOL (Anhui) Co., Ltd), and expressed and purified on HEK293E cells according to the method described in Example 2.1. The sequence information of the control antibodies is referring to Table 4.

TABLE 4
Sequence information of the control antibody

| Sequence name | |
|---|---|
| and No. | Amino acid sequence |
| H65 VH | NIQLVQSGPELKKPGETVKISCKASGYTFTNYGMNWVKQAPGKGLRWM |
| (SEQ ID NO: 117) | GWINTHTGEPTYADDFKGRFAFSLETSASTAYLQINNLKNEDTATYFCTRR |
| | GYDWYFDVWGAGTTVTVSS |
| H65 VL | DIKMTQSPSSMYASLGERVTITCKASQDINSYLSWFHHKPGKSPKTLIYRA |
| (SEQ ID NO: 118) | NRLVDGVPSRFSGSGSGQDYSLTISSLDYEDMGIYYCQQYDESPWTFGGG |
| | TKLEMK |
| H65 scFv-hFc | NIQLVQSGPELKKPGETVKISCKASGYTFTNYGMNWVKQAPGKGLRWM |
| (SEQ ID NO: 119) | GWINTHTGEPTYADDFKGRFAFSLETSASTAYLQINNLKNEDTATYFCTRR |
| | GYDWYFDVWGAGTTVTVSSGGGGSGGGGSGGGGSDIKMTQSPSSMYAS |
| | LGERVTITCKASQDINSYLSWFHHKPGKSPKTLIYRANRLVDGVPSRFSGS |
| | GSGQDYSLTISSLDYEDMGIYYCQQYDESPWTFGGGTKLEMKEPKSADK |
| | THTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQQGN |
| | VFSCSVMHEALHNHYTQKSLSLSPGK |

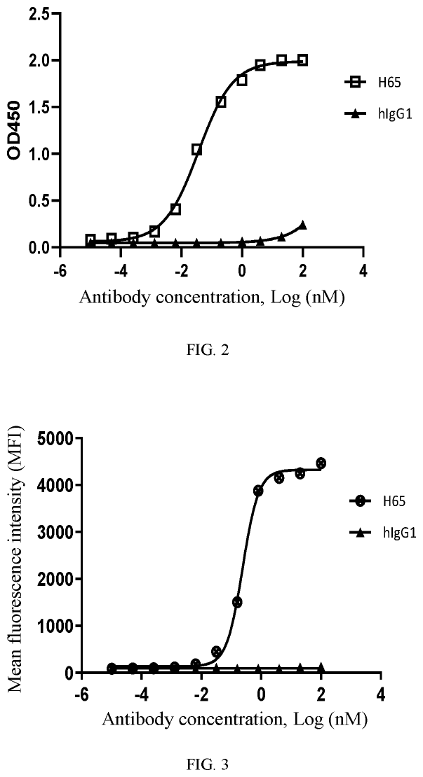

The binding activity of the H65 antibody prepared above with human CD5 protein was subjected to ELISA assay. A purchased commercial antigen (human CD5-His) was diluted with PBS to obtain a final concentration of 2 μg/mL, and then added to a 96-well ELISA plate with 100 L/well. The plate was sealed with a plastic film and incubated at 4° C. over the night, washed twice with PBS in the next day, added with a blocking solution [PBS+2% (w/w) BSA] and blocked at room temperature for 2 h. The blocking solution was drained out, and 100 nM of the H65 antibody or negative control antibody hIgG1 after gradient dilution was added, namely, the obtained product was the antibody Anti-HEL-hIgG1 against hen egg lysozyme (purchased from Biointron, Cat. No. B117901) with 50 μl per well. After incubation for 2 h at 37° C., the plate was washed for 3 times with PBS. A secondary antibody labeled with HRP (horseradish peroxidase) (A0170, purchased from Sigma) was added. After incubation for 2 h at 37° C., the plate was washed for 5 times with PBS. A TMB substrate was added with 50 μL/well and incubated at room temperature for 30 min, then a stop solution (1.0 N HCl) was added with 50 μL/well. The OD_{450 nm} value was read by an ELISA plate reader (Multimode Plate Reader, EnSight, purchased from Perkin Elmer), as shown in FIG. 2., H65 has good binding activity with human CD5 protein.

### Example 3. Identification of Cell Strain Endogenously Expressing CD5 and Preparation of Over-Expression Cell Strain

### 3.1 Identification of the Cell Strain Endogenously Expressing CD5

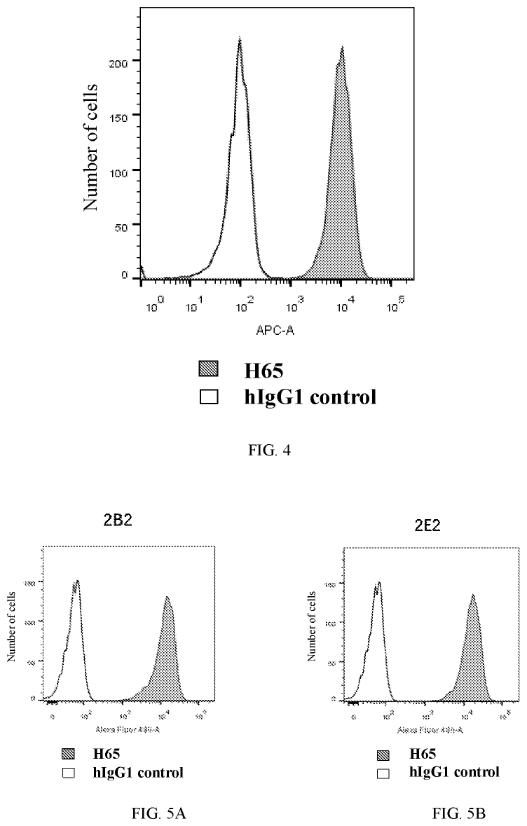

Jurkat (ATCC, TIB-152) cells were expanded to a logarithmic growth phase in a T-25 cell culture flask, the culture supernatant was discarded by centrifugation, and the cell pellet was washed twice with PBS. The results were assayed and analyzed by FACS (FACS Canto™, purchased from BD Biosciences) using the antibody H65 as a primary antibody (working concentration: 100 nM) and an Alexa Flour488-labeled secondary antibody (purchased from Invitrogen, Cat. No. A11013). The analysis results are shown in Table 5 and FIGS. 3-4, which show that Jurkat cells bind to H65.

TABLE 5
Assay results for Jurkat cells of the endogenous cell line by FACS

Mean fluorescence intensity of cells

| No. | Antibody name | IgG subtype control | CD5 antibody |
|---|---|---|---|
| 1 | H65 | 97 | 9892 |

3.2 Preparation of CHO-K1 Monoclonal Cell Strain Stably Transfected with Human CD5

A nucleotide sequence encoding a full-length amino acid sequence of humanized CD5 (NCBI: NP_055022.2) was cloned into a pcDNA3.1 vector (purchased from Thermofisher scientific), and a plasmid was prepared. After transfection of the cell line CHO-K1 (purchased from National Collection of Authenticated Cell Cultures of Chinese Academy of Sciences) with a plasmid (Lipofectamine® 3000 Transfection Kit, purchased from Invitrogen, Cat. No. L3000-015), the cells were selectively cultured for 2 weeks in a DMEM/F12 medium containing 10 μg/ml puromycin and 10% (w/w) fetal bovine serum. Positive monoclonal cells were sorted on a flow cytometer FACSAraiII (BD Biosciences) with the H65 antibody as a primary antibody and an Alexa Flour488-labeled secondary antibody (Invitrogen, Cat. No. A11030) onto a 96-well plate, and then cultured at 37° C. with 5% (v/v) CO₂; about 2 weeks later, some of monoclonal wells were selected and amplified. The amplified clones were screened by flow cytometry. Monoclonal cell lines with better growth and higher fluorescence intensity were selected for further expansion and cryopreserved in liquid nitrogen.

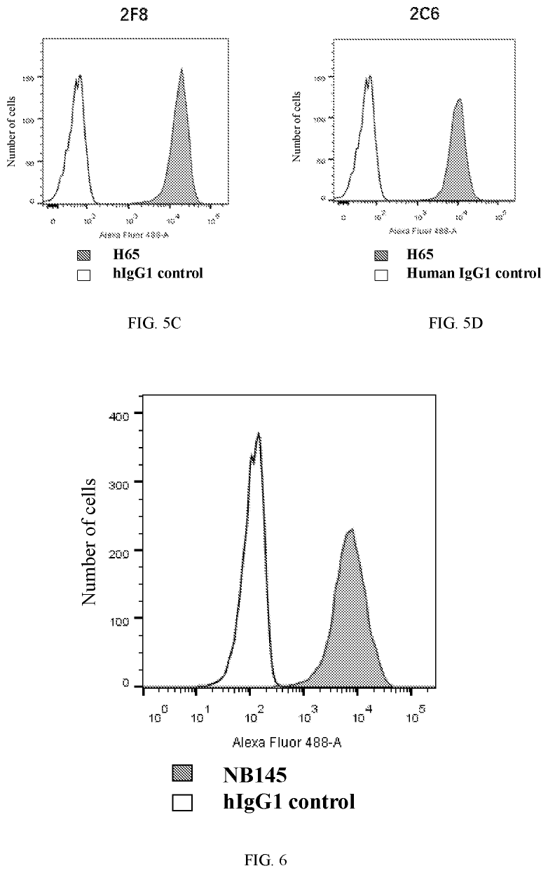

The specific results for the selection are shown in Table 6 and FIGS. 5A-5D, in which the IgG subtype control is a human IgG1 control. Table 6 indicates that a series of CHO-K1 monoclonal cell lines with positive expression of human CD5 (namely, hCD5) have been prepared. In FIGS. 5A-5D, x-coordinate denotes the fluorescence intensity of cells, and y-coordinate denotes the number of cells. Results of the FIGS. 5A-5D indicate that CHO-K1-hCD5-2B2, CHO-K1-hCD5-2E2, CHO-K1-hCD5-2F8 and CHO-K1-hCD5-2C6 are cell strains with high level expression of hCD5. CHO-K1-hCD5-2B2 was selected as the stably transfected cell line used in the subsequent experiment.

TABLE 6
Assay results for CHO-K1 cell line stably transfected
with the human CD5 protein by FACS

| | | Mean fluorescence intensity of cells | |
|---|---|---|---|
| | Clone No. of stably | IgG subtype | |
| No. | transfected cell line | control | CD5 antibody |
| 1 | CHO-K1-hCD5-2B2 | 65.9 | 12975 |
| 2 | CHO-K1-hCD5-2E2 | 65.9 | 16129 |
| 3 | CHO-K1-hCD5-2F8 | 65.9 | 10178 |
| 4 | CHO-K1-hCD5-2C6 | 65.9 | 12975 |

3.3 Preparation of HEK293T Cell Strain Stably Transfected with Monkey CD5

A nucleotide sequence encoding a full-length amino acid sequence of monkey CD5 (NCBI: H9ZFB2-1) was cloned into a pcDNA3.1 vector (purchased from Thermofisher scientific), and a plasmid was prepared.

After plasmid transfection of the HEK293T cell line with FuGENE® HD (Promega, Cat. No. E2311), the cells were selectively cultured in a DMEM medium containing 10 μg/mL puromycin and 10% (w/w) fetal bovine serum for 2 weeks, subcloned in a 96-well culture plate by a limiting dilution method, and cultured at 37° C. with 5% (v/v) CO₂. About 2 weeks later, some of the polyclonal wells were selected and amplified in a 6-well plate. The amplified clones were screened by flow cytometry using an NB145 (serum obtained after the Vicugna pacos was immunized by human CD5 protein, which had been verified to be bound to monkey CD5 protein) polyclonal antibody, and the cell line with better growth and higher fluorescence intensity was selected as the HEK293T stably transfected cell strain for further expansion and cryopreserved in liquid nitrogen. FIG. 6 shows that the HEK293T stably transfected cell strain has a single positive cell peak overexpressing monkey CD5 after puromycin screening, and may be used for detecting the cross-activity of the antibodies.

TABLE 7
Table for amino acid sequences of full-length human/monkey CD5

| Protein | Amino acid sequence |
|---|---|
| Human CD5 | MGSLQPLATLYLLGMLVASCLGRLSWYDPDFQARLTRSNSKCQGQLEVYL |
| (SEQ ID NO: | KDGWHMVCSQSWGRSSKQWEDPSQASKVCQRLNCGVPLSLGPFLVTYTP |
| 120) | QSSIICYGQLGSFSNCSHSRNDMCHSLGLTCLEPQKTTPPTTRPPPTTTPEPTA |
| | PPRLQLVAQSGGQHCAGVVEFYSGSLGGTISYEAQDKTQDLENFLCNNLQC |
| | GSFLKHLPETEAGRAQDPGEPREHQPLPIQWKIQNSSCTSLEHCFRKIKPQK |
| | SGRVLALLCSGFQPKVQSRLVGGSSICEGTVEVRQGAQWAALCDSSSARSS |
| | LRWEEVCREQQCGSVNSYRVLDAGDPTSRGLFCPHQKLSQCHELWERNSY |
| | CKKVFVTCQDPNPAGLAAGTVASIILALVLLVVLLVVCGPLAYKKLVKKFR |
| | QKKQRQWIGPTGMNQNMSFHRNHTATVRSHAENPTASHVDNEYSQPPRNS |
| | HLSAYPALEGALHRSSMQPDNSSDSDYDLHGAQRL |
| Monkey CD5 | MPMRSPQPLATLYLLGMLVASCLGRLSWDDPDFQTRLTRSNSRCQGQLEVY |
| (SEQ ID NO: | IKYGWHMVCSQSWGRSSNQWEDPNQASKVCQRLNCGVPLSLGPFLITDRH |
| 121) | QSQITCYGRLGSFSNCSHSGRDVCRPLGLTCLEPQTTTPPPTRPPPTTTPEPTA |
| | PPRLOLVAQAAGRHCAGVVEFYSGSLGGTISYEAQDKAQDKTQVLENFLCS |
| | SLQCGSFLKHLPETEAATAQDPGELREHQPLPIQWTIRNSSCTSLEHCFRKIK |
| | PRNSGQVLALLCSGFQPKVQSRLVGGSSICEGTVEVRQGAQWAALCDSSSA |
| | KSSLRWEEVCQEQQCGSFNSYQALDAGDPTSRGLSCPHQKLSQCHELTERK |
| | SYCKKVFVTCQDPNPAGPAAGTVASIILALVLLGVLLVVCGPLAYKKLVKKF |
| | RQKKQRQWIGPTGMNQNMSFHRNHTATVRSHVENPTASHVDNEYSQPPRN |
| | SRLSAYPALEGALHRSSTQPDNSSDSDYDLHGAQRL |

### Example 4. Identification of VHH-hFc

### 4.1 Assay on Binding of VHH-hFc to Human CD5 Protein by Enzyme-Linked Immunosorbent Assay (ELISA)

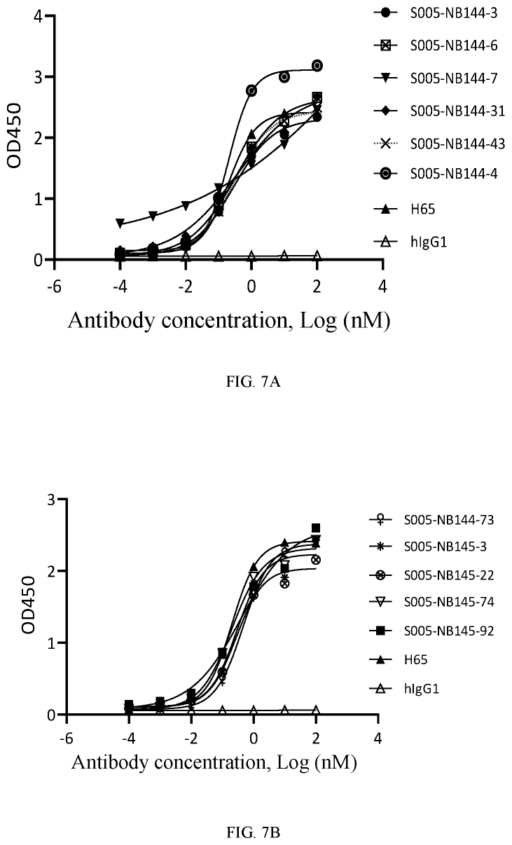

To assay the binding activity of VHH-hFc to human CD5 protein, ELISA assay was performed according to the method described in Example 2.2. The assay results for the binding of VHH-hFc to hCD5-His by ELISA are shown in FIGS. 7A-7B and Table 8. Table 8 indicates that all the purified antibodies may bind to hCD5-His at ELISA level. IgG control is hIgG1, and data in the table are values of OD_{450 nm}.

TABLE 8
Assay results for binding reactions of VHH-hFc
antibodies with human CD5 protein by ELISA

OD450

| Antibody | Antibody concentration (nM) | | | | | | | |
|---|---|---|---|---|---|---|---|---|
| name | 100 | 10 | 1 | 0.1 | 0.01 | 0.001 | 0.0001 | blank |
| S005-NB144-3 | 2.34 | 2.05 | 1.70 | 0.79 | 0.21 | 0.12 | 0.09 | 0.07 |
| S005-NB144-6 | 2.66 | 2.28 | 1.84 | 0.90 | 0.26 | 0.14 | 0.11 | 0.06 |
| S005-NB144-7 | 2.45 | 1.89 | 1.55 | 1.17 | 0.88 | 0.71 | 0.59 | 0.06 |
| S005-NB144-31 | 2.67 | 2.07 | 1.84 | 1.02 | 0.39 | 0.21 | 0.16 | 0.06 |
| S005-NB144-43 | 2.50 | 2.16 | 1.84 | 0.79 | 0.24 | 0.12 | 0.09 | 0.06 |
| S005-NB144-73 | 2.40 | 2.24 | 1.65 | 0.48 | 0.14 | 0.08 | 0.07 | 0.06 |
| S005-NB145-3 | 2.43 | 1.91 | 1.83 | 0.61 | 0.20 | 0.13 | 0.10 | 0.06 |
| S005-NB145-22 | 2.16 | 1.83 | 1.67 | 0.59 | 0.20 | 0.12 | 0.09 | 0.05 |
| S005-NB145-74 | 2.43 | 2.08 | 1.92 | 0.85 | 0.22 | 0.10 | 0.08 | 0.06 |
| S005-NB145-92 | 2.60 | 2.04 | 1.78 | 0.87 | 0.30 | 0.18 | 0.14 | 0.06 |
| S005-NB144-4 | 3.18 | 3.00 | 2.77 | 1.01 | 0.28 | 0.17 | 0.09 | 0.05 |
| H65 | 2.82 | 2.35 | 1.87 | 0.64 | 0.17 | 0.09 | 0.07 | 0.12 |
| hIgG1 | 0.06 | 0.05 | 0.05 | 0.05 | 0.05 | 0.05 | 0.05 | 0.06 |

### 4.2 Assay on Binding of Antibodies to Human CD5-Expressing Cells by Flow Cytometry Assay (FACS)

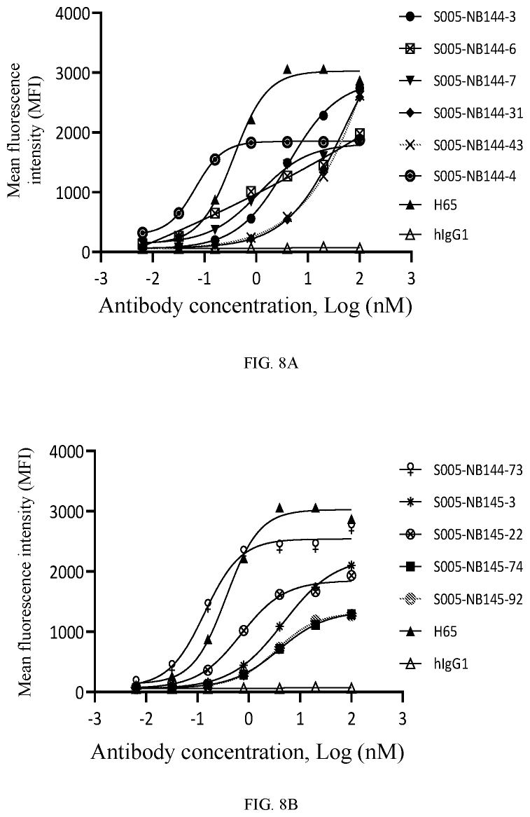

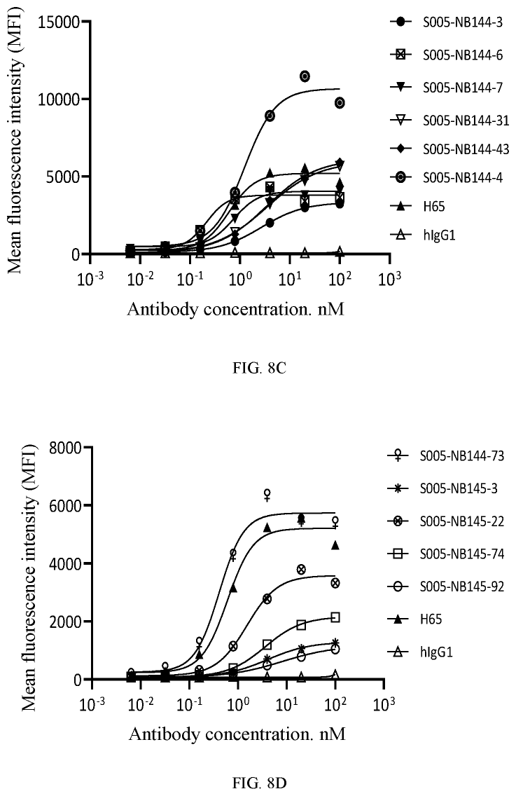

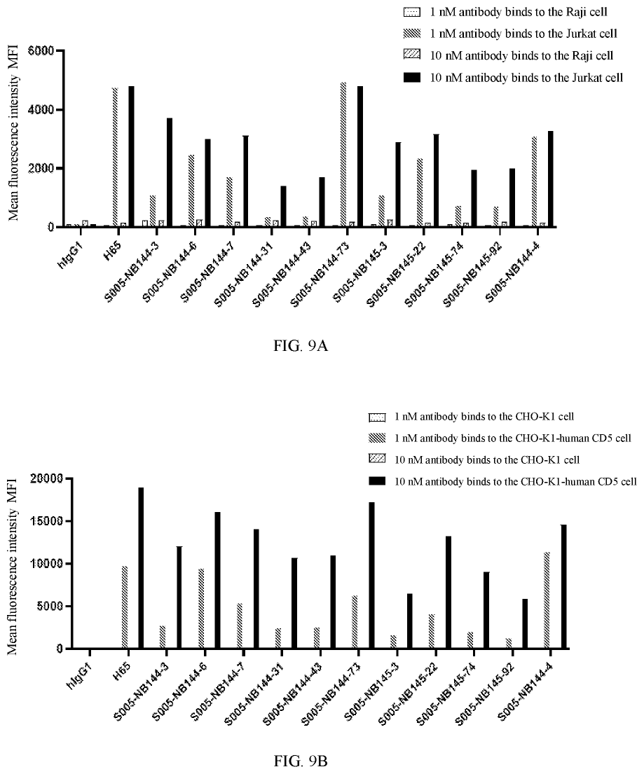

The desired cells were expanded to a logarithmic growth phase in a T-75 cell culture flask. Adherent cells CHO-K1-human CD5 were removed from the culture medium by pipetting, and the cells were washed twice with a PBS buffer, digested with trypsin, and washed twice with a PBS buffer again after the digestion was stopped. For suspension cells Jurkat, the medium supernatant was discarded by direct centrifugation, and the cell pellet was washed twice with PBS. After the cells obtained in the last step were subjected to cell counting, the cell pellet was resuspended to 2×10⁶ cells/mL with a blocking solution [PBS+2% (w/w) BSA] and added to a 96-well FACS reaction plate with 50 μL/well. The VHH-hFc or control antibody was added with 50 μL/well, and the plate was incubated on ice for 2 h. After the plate was centrifuged and washed for 3 times with a PBS buffer, an Alexa Fluor 488-labeled secondary antibody (A-11013, purchased from Invitrogen) was added with 50 μL/well, and the plate was incubated on ice for 1 h. After the plate was centrifuged and washed for 5 times with a PBS buffer, the results were assayed and analyzed by FACS (FACS Canto™, purchased from BD Biosciences). Data analysis was performed by software (BD FACSDiva) to obtain the mean fluorescence intensity (MFI) of the cells. Analysis was then performed by software (GraphPad Prism8), data were fitted, and EC₅₀ values were calculated. The analysis results are shown in Table 9 and FIGS. 8A-8D, and show that all the VHH-hFc may bind to the human CD5 protein on the surface of Jurkat cells and CHO-K1-human CD5 cells. The same method was used to assay the binding reactions of 1 nM and 10 nM VHH-hFc antibodies with Jurkat cells and endogenous CD5-negative Raji cells, and to assay the binding reactions of 1 nM and 10 nM VHH-hFc antibodies with CHO-K1 cells and CHO-K1-human CD5. The results are shown in FIGS. 9A-9B, showing that all the VHH-hFc do not bind to Raji cells and CHO-K1 cells and thus, have good specificity.

TABLE 9
Assay results for binding reactions of VHH-hFc
with Jurkat and CHO-K1-human CD5 cells by FACS

| | Jurkat | | CHO-K1-human CD5 | |
|---|---|---|---|---|
| | Maximum mean | | Maximum mean | |
| | fluorescence | | fluorescence | |
| | intensity | | intensity | |
| Antibody name | Max MFI | EC50(nM) | Max MFI | EC50(nM) |
| S005-NB144-3 | 2742 | 4.09 | 3243 | 2.71 |
| S005-NB144-6 | 1981 | Certain binding, | 3662 | 0.21 |
| | | but poor fitting | | |
| S005-NB144-7 | 1850 | 1.11 | 4089 | 0.75 |
| S005-NB144-31 | 2590 | 51.01 | 5685 | 3.47 |
| S005-NB144-43 | 2618 | 427.20 | 5924 | 3.47 |
| S005-NB144-73 | 2729 | 0.14 | 5392 | 0.41 |
| S005-NB145-3 | 2103 | 4.70 | 1260 | 4.08 |
| S005-NB145-22 | 1934 | 0.71 | 3326 | 1.53 |
| S005-NB145-74 | 1290 | 3.85 | 2140 | 3.64 |
| S005-NB145-92 | 1277 | 3.46 | 1047 | 8.22 |
| S005-NB144-4 | 1874 | 0.08 | 9751 | 1.19 |
| H65 | 2749 | 0.32 | 4699 | 0.80 |
| hIgG1 | 62 | Negative | 93 | Negative |

### Example 5. Assay on Cross-Binding Activity of VHH-hFc

5.1 Assay on Binding of VHH-hFc with a Murine CD5 Protein by ELISA

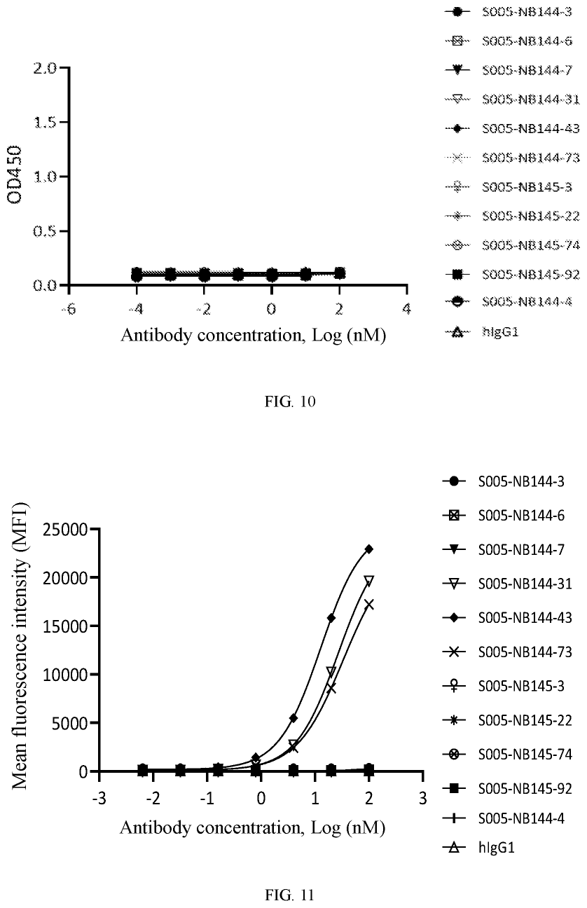

To assay the species cross-activity of the VHH-hFc, ELISA plates were coated with commercial murine CD5-His (CD5 (Sino Biologicals, Cat. No. 50403-M08H)), and ELISA assays were performed according to the method described in Example 2.2. The assay results for the binding of VHH-hFc to murine CD5 by ELISA are shown in FIG. 10 and Table 10. Table 10 indicates that the purified antibodies do not bind to murine CD5 at the ELISA level. The negative control is hIgG1 and NB145 serves as the positive control (NB145 has been verified to be bound to murine CD5); data in the table are values of OD_{450 nm}.

TABLE 10
Assay results for binding reactions of VHH-hFc
antibodies with murine CD5 protein by ELISA

OD450

| Antibody | Antibody concentration (nM) | | | | | | | |
|---|---|---|---|---|---|---|---|---|
| name | 100.00 | 10.00 | 1.00 | 0.10 | 0.01 | 0.001 | 0.000 | blank |
| S005-NB144-3 | 0.11 | 0.10 | 0.11 | 0.10 | 0.09 | 0.11 | 0.11 | 0.11 |
| S005-NB144-6 | 0.11 | 0.09 | 0.10 | 0.09 | 0.09 | 0.09 | 0.09 | 0.10 |
| S005-NB144-7 | 0.12 | 0.10 | 0.09 | 0.09 | 0.09 | 0.09 | 0.09 | 0.11 |
| S005-NB144-31 | 0.11 | 0.10 | 0.10 | 0.10 | 0.09 | 0.10 | 0.09 | 0.10 |
| S005-NB144-43 | 0.11 | 0.10 | 0.11 | 0.10 | 0.09 | 0.10 | 0.10 | 0.10 |
| S005-NB144-73 | 0.11 | 0.10 | 0.10 | 0.10 | 0.09 | 0.10 | 0.10 | 0.11 |
| S005-NB145-3 | 0.11 | 0.09 | 0.08 | 0.09 | 0.09 | 0.09 | 0.09 | 0.10 |
| S005-NB145-22 | 0.11 | 0.11 | 0.09 | 0.09 | 0.09 | 0.09 | 0.09 | 0.10 |
| S005-NB145-74 | 0.12 | 0.12 | 0.12 | 0.12 | 0.13 | 0.12 | 0.13 | 0.14 |
| S005-NB145-92 | 0.12 | 0.11 | 0.11 | 0.11 | 0.11 | 0.12 | 0.12 | 0.12 |
| S005-NB144-4 | 0.11 | 0.09 | 0.08 | 0.09 | 0.08 | 0.09 | 0.08 | 0.09 |
| hIgG1 | 0.12 | 0.12 | 0.11 | 0.11 | 0.11 | 0.11 | 0.11 | 0.11 |

| | | |
|---|---|---|
| NB145 | 1.52 | * The antibody is a polyclonal antibody; the |
| (1:100) | | reaction concentration is a concentration after |
| | | being diluted by 1:100 |

5.2 Assay on Binding of VHH-hFc to the Expressing Cell Lines Stably Transfected with Monkey CD5 by FACS

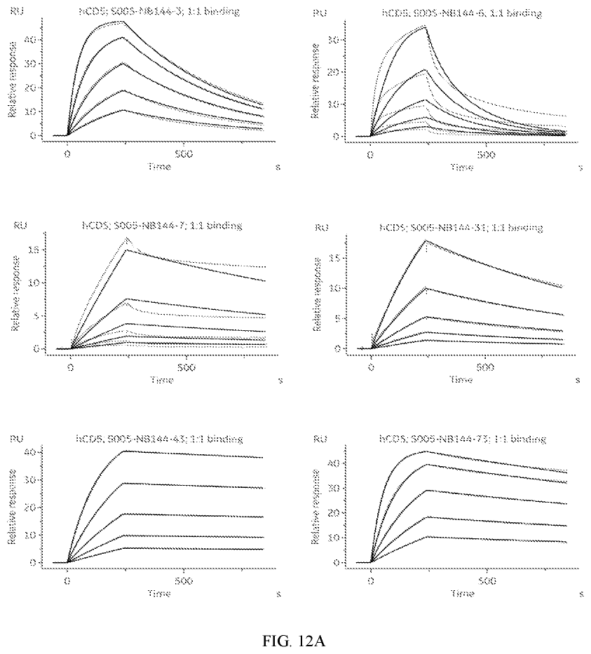

HEK293T-monkey CD5 cells were subjected to the FACS assay and data analysis according to the method described in Example 4.2. The analysis results are shown in Table 11 and FIG. 11, indicating that only S002-NB144-31, S002-NB144-43 and S002-NB144-73 have binding activity to the expressing cell lines stably transfected with monkey CD5. The negative control is hIgG1 and NB145 serves as the positive control (the dilution of NB145 is 1:100).

TABLE 11
Assay results for binding reactions of VHH-hFc antibodies
with HEK293T-monkey CD5 cells by FACS

HEK293T-monkey CD5

| | Maximum mean | |
|---|---|---|
| | fluorescence | |
| | intensity | |
| Antibody name | Max MFI | EC50 (nM) |
| S005-NB144-3 | 76 | Negative |
| S005-NB144-6 | 150 | Negative |
| S005-NB144-7 | 102 | Negative |
| S005-NB144-31 | 19619 | 26.99 |
| S005-NB144-43 | 22930 | 12.78 |
| S005-NB144-73 | 17218 | 33.21 |
| S005-NB145-3 | 110 | Negative |
| S005-NB145-22 | 114 | Negative |
| S005-NB145-74 | 76 | Negative |
| S005-NB145-92 | 198 | Negative |
| S005-NB144-4 | 75 | Negative |
| NB145 | 4308 | Positive |
| hIgG1 | 79 | Negative |

### Example 6. Assay on Affinity of CD5 Antibody

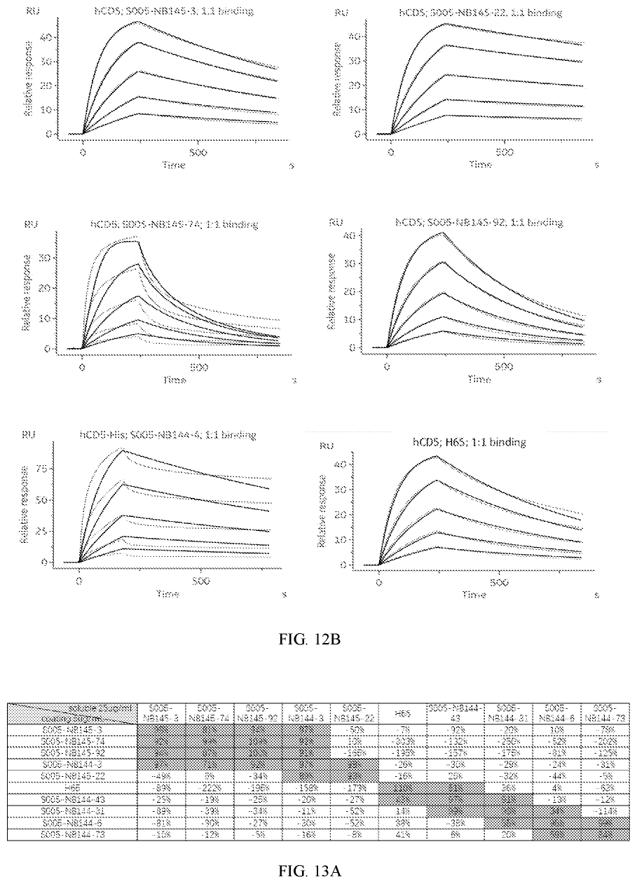

The anti-human CD5 VHH-hFc was captured using a Protein A chip (GE Healthcare; 29-127-558). The sample and running buffer were HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the test antibody was captured using the Protein A chip, and then a single concentration of CD5 antigen protein was injected. The association and dissociation processes of the antibody with the antigen protein were recorded, and finally, the chip was regenerated using Glycine pH 1.5 (GE Healthcare; BR-1003-54). The association was determined by injecting different concentrations of recombinant human CD5-His in the solution and maintaining for 240 s, wherein the flow rate was 30 μL/min, and the protein was diluted in a 1:1 dilution ratio from 200 nM (see detailed results for actual concentrations tested) to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface, and blank injections were also subtracted (=double referencing). To calculate the apparent KD and other kinetic parameters, the Langmuir 1:1 model was used. The association rate (Kon), dissociation rate (Koff), and binding affinity (KD) of the VHH-hFc with the hCD5-His protein are shown in Table 12, in which the antibody H65 is used as a control. As shown in FIGS. 12A-12B and Table 12, the affinity of VHH-hFc antibodies with human CD5 is less than 1E-6 M, and all the affinity are less than 1E-7 M except S005-NB144-7; further, the affinity is less than 1E-8 M except S005-NB144-6, S005-NB144-31 and S005-NB145-74; whereas S005-NB144-43, S005-NB144-73 and S005-NB145-22 have good binding to human CD5, and all the affinity thereof are less than 1E-9 M.

TABLE 12
Assay on affinity of VHH-hFc antibodies
to human CD5 by BIAcore

| Antibody name | ka (1/Ms) | kd (1/s) | KD (M) |
|---|---|---|---|
| S005-NB144-3 | 7.81E+05 | 2.18E−03 | 2.80E−09 |
| S005-NB144-6 | 6.74E+05 | 2.87E−02 | 4.26E−08 |
| S005-NB144-7 | 4.57E+03 | 6.80E−04 | 1.49E−07 |
| S005-NB144-31 | 4.11E+04 | 9.61E−04 | 2.34E−08 |
| S005-NB 144-43 | 3.04E+05 | 9.87E−05 | 3.25E−10 |
| S005-NB144-73 | 1.41E+06 | 3.51E−04 | 2.48E−10 |
| S005-NB145-3 | 5.36E+05 | 9.22E−04 | 1.72E−09 |
| S005-NB145-22 | 1.02E+06 | 3.63E−04 | 3.55E−10 |
| S005-NB 145-74 | 1.13E+06 | 1.46E−02 | 1.29E−08 |
| S005-NB145-92 | 4.26E+05 | 2.40E−03 | 5.64E−09 |
| S005-NB144-4 | 1.91E+05 | 7.01E−04 | 3.67E−09 |
| H65 | 9.47E+05 | 1.48E−03 | 1.56E−09 |

### Example 7 Analysis on Antibody-Antigen Binding Epitopes

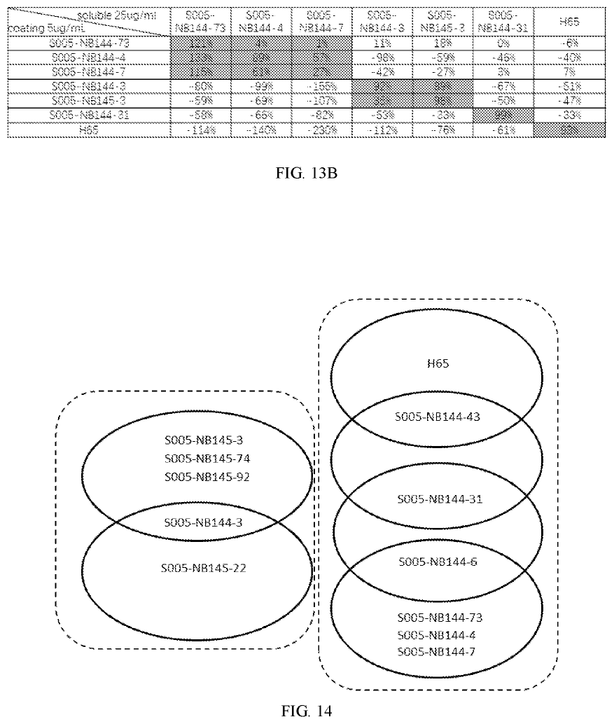

VHH-hFc was subjected to epitope classification with competitive ELISA. ELISA plates were coated with 2 μg/mL antibodies according to the method described in Example 4.1; the human CD5-his protein was subjected to a gradient dilution from 30 μg/mL, and EC₈₀ values (Table 13) were calculated and applied to the next step of epitope analysis. ELISA plates were coated with 2 μg/mL antibodies; after 25 μg/mL of antibodies to be tested were added, a hCD5-his protein having a EC₈₀ concentration corresponding to each of the antibodies to be tested was then added for incubation for 2 h; the plates were washed for 5 times with PBS and HRP-labeled anti-His antibodies (GenScript, Cat. No. A00612) were added for assay. The coated antibody may bind to the antibody-to be tested-hCD5-his antigen complex in the solution if no competitive relation exists between the coated antibody and the antibody-to be tested in the solution. Moreover, OD_{450 nm} absorbance was detected and an inhibition ratio between each pair of antibodies was calculated according to the OD_{450 nm} absorbance (FIGS. 13A-3B).

Epitopes of each antibody were classified as shown in FIG. 14 according to the inhibition ratio. A competitive relation exists between S005-NB144-43 and H65, indicating that S005-NB144-43 and H65 are of the same category; meanwhile, a competitive relation exists between S005-NB144-43 and S005-NB144-31, but there is no competitive relation between S005-NB144-31 and H65; meanwhile, a competitive relation exists between S005-NB144-31 and S005-NB144-6, and a competitive relation exists between S005-NB144-6 and S005-NB144-73 at the same time; moreover, there is a competitive relation between S005-NB144-73 and S005-NB144-4 as well as between S005-NB144-73 and S005-NB144-7; therefore, the above antibodies may be classified into a category. There are 5 antibodies without any competition with H65, of which S005-NB145-3, S005-NB145-7 and S005-NB145-92 are competitive with S005-NB144-3, and meanwhile, S005-NB144-3 is also competitive with S005-NB145-22; therefore, these antibodies may be classified into a category.

TABLE 13
EC₈₀ values of the human CD5 protein corresponding to VHH-hFc

| Antibody name | EC₈₀ (μg/mL) |
|---|---|
| S005-NB144-3 | 0.15 |
| S005-NB144-6 | 1.59 |
| S005-NB144-7 | 0.67 |
| S005-NB144-31 | 0.11 |
| S005-NB144-43 | 0.03 |
| S005-NB144-73 | 0.02 |
| S005-NB145-3 | 0.07 |
| S005-NB145-22 | 0.06 |
| S005-NB145-74 | 0.59 |
| S005-NB145-92 | 0.23 |
| S005-NB 144-4 | 0.10 |
| H65 | 0.25 |

The CD5 antibody and use thereof provided herein have been described above in detail. The principle and embodiments of the present disclosure are described with specific examples herein. The above examples are merely intended to help understanding the method and core idea of the present disclosure. It should be pointed out that those skilled in the art can further make several improvements and modifications to the present disclosure, without departing from the principle of the present disclosure; moreover, these improvements and modifications also fall within the protection scope of the claims of the present disclosure.

1. An antibody or an antigen-binding fragment specifically binding to CD5, wherein the antibody or the antigen-binding fragment comprises a CDR1, a CDR2, and a CDR3; the CDR1, the CDR2, and the CDR3 comprise an HCDR1, an HCDR2, and an HCDR3 selected from a VHH domain set forth in any one of SEQ ID NOs: 6-16; or
the CDR1, the CDR2, and/or the CDR3 comprise amino acid sequences having at most 10, 9, 8, 7, 6, 5, 4, 3, 2, or 1 mutation on the HCDR1, the HCDR2 and/or the HCDR3; the mutation may be selected from an insertion, a deletion, and/or a substitution; the substitution is preferably a substitution of conserved amino acids.

2. The antibody or the antigen-binding fragment according to claim 1, wherein the HCDR1, the HCDR2, and the HCDR3 are determined according to the IMGT numbering scheme, the Kabat numbering scheme, or the Chothia numbering scheme; optionally, the HCDR1, the HCDR2, and the HCDR3 are selected from Table 3;
optionally, the HCDR1 has an amino acid sequence set forth in SEQ ID NO: 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, or 113;
optionally, the HCDR2 has an amino acid sequence set forth in SEQ ID NO: 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, or 114;
optionally, the HCDR3 has an amino acid sequence set forth in SEQ ID NO: 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, or 115; or
the HCDR1, the HCDR2, and the HCDR3 have amino acid sequences set forth in SEQ ID NOs: 17-19 according to the IMGT numbering scheme; or
the HCDR1, the HCDR2, and the HCDR3 have amino acid sequences set forth in SEQ ID NOs: 20-22 according to the Kabat numbering scheme; or
the HCDR1, the HCDR2, and the HCDR3 have amino acid sequences set forth in SEQ ID NOs: 23-25 according to the Chothia numbering scheme; or
the HCDR1, the HCDR2, and the HCDR3 have amino acid sequences set forth in SEQ ID NOs: 26-28 according to the IMGT numbering scheme; or
the HCDR1, the HCDR2, and the HCDR3 have amino acid sequences set forth in SEQ ID NOs: 29-31 according to the Kabat numbering scheme; or
the HCDR1, the HCDR2, and the HCDR3 have amino acid sequences set forth in SEQ ID NOs: 32-34 according to the Chothia numbering scheme; or
the HCDR1, the HCDR2, and the HCDR3 have amino acid sequences set forth in SEQ ID NOs: 35-37 according to the IMGT numbering scheme; or
the HCDR1, the HCDR2, and the HCDR3 have amino acid sequences set forth in SEQ ID NOs: 38-40 according to the Kabat numbering scheme; or
the HCDR1, the HCDR2, and the HCDR3 have amino acid sequences set forth in SEQ ID NOs: 41-43 according to the Chothia numbering scheme; or
the HCDR1, the HCDR2, and the HCDR3 have amino acid sequences set forth in SEQ ID NOs: 44-46 according to the IMGT numbering scheme; or
the HCDR1, the HCDR2, and the HCDR3 have amino acid sequences set forth in SEQ ID NOs: 47-49 according to the Kabat numbering scheme; or
the HCDR1, the HCDR2, and the HCDR3 have amino acid sequences set forth in SEQ ID NOs: 50-52 according to the Chothia numbering scheme; or
the HCDR1, the HCDR2, and the HCDR3 have amino acid sequences set forth in SEQ ID NOs: 53-55 according to the IMGT numbering scheme; or
the HCDR1, the HCDR2, and the HCDR3 have amino acid sequences set forth in SEQ ID NOs: 56-58 according to the Kabat numbering scheme; or
the HCDR1, the HCDR2, and the HCDR3 have amino acid sequences set forth in SEQ ID NOs: 59-61 according to the Chothia numbering scheme; or
the HCDR1, the HCDR2, and the HCDR3 have amino acid sequences set forth in SEQ ID NOs: 62-64 according to the IMGT numbering scheme; or
the HCDR1, the HCDR2, and the HCDR3 have amino acid sequences set forth in SEQ ID NOs: 65-67 according to the Kabat numbering scheme; or
the HCDR1, the HCDR2, and the HCDR3 have amino acid sequences set forth in SEQ ID NOs: 68-70 according to the Chothia numbering scheme; or
the HCDR1, the HCDR2, and the HCDR3 have amino acid sequences set forth in SEQ ID NOs: 71-73 according to the IMGT numbering scheme; or
the HCDR1, the HCDR2, and the HCDR3 have amino acid sequences set forth in SEQ ID NOs: 74-76 according to the Kabat numbering scheme; or
the HCDR1, the HCDR2, and the HCDR3 have amino acid sequences set forth in SEQ ID NOs: 77-79 according to the Chothia numbering scheme; or
the HCDR1, the HCDR2, and the HCDR3 have amino acid sequences set forth in SEQ ID NOs: 80-82 according to the IMGT numbering scheme; or
the HCDR1, the HCDR2, and the HCDR3 have amino acid sequences set forth in SEQ ID NOs: 83-85 according to the Kabat numbering scheme; or
the HCDR1, the HCDR2, and the HCDR3 have amino acid sequences set forth in SEQ ID NOs: 86-88 according to the Chothia numbering scheme; or
the HCDR1, the HCDR2, and the HCDR3 have amino acid sequences set forth in SEQ ID NOs: 89-91 according to the IMGT numbering scheme; or
the HCDR1, the HCDR2, and the HCDR3 have amino acid sequences set forth in SEQ ID NOs: 92-94 according to the Kabat numbering scheme; or
the HCDR1, the HCDR2, and the HCDR3 have amino acid sequences set forth in SEQ ID NOs: 95-97 according to the Chothia numbering scheme; or
the HCDR1, the HCDR2, and the HCDR3 have amino acid sequences set forth in SEQ ID NOs: 98-100 according to the IMGT numbering scheme; or
the HCDR1, the HCDR2, and the HCDR3 have amino acid sequences set forth in SEQ ID NOs: 101-103 according to the Kabat numbering scheme; or
the HCDR1, the HCDR2, and the HCDR3 have amino acid sequences set forth in SEQ ID NOs: 104-106 according to the Chothia numbering scheme; or
the HCDR1, the HCDR2, and the HCDR3 have amino acid sequences set forth in SEQ ID NOs: 107-109 according to the IMGT numbering scheme; or
the HCDR1, the HCDR2, and the HCDR3 have amino acid sequences set forth in SEQ ID NOs: 110-112 according to the Kabat numbering scheme; or
the HCDR1, the HCDR2, and the HCDR3 have amino acid sequences set forth in SEQ ID NOs: 113-115 according to the Chothia numbering scheme.

3. (canceled)

4. The antibody or the antigen-binding fragment according to claim 1, wherein the CDR1, the CDR2, and/or the CDR3 comprise sequences having at least 80%, 85%, 90%, 91%, 92%, 93%, 94%, 95%, 96%, 97%, 98%, 99%, or 100% identity to the HCDR1, the HCDR2, and/or the HCDR3, respectively.

5. The antibody or the antigen-binding fragment according to claim 1, wherein the antibody or the antigen-binding fragment comprises a single-domain antibody comprising the CDR1, the CDR2, and the CDR3; or
the single-domain antibody comprises a sequence set forth in any one of SEQ ID NOs: 6-16; or
the single-domain antibody comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 6-16; the mutation may be selected from an insertion, a deletion, and/or a substitution; the substitution is preferably a substitution of conserved amino acids; or
the single-domain antibody comprises a sequence having at least 80%, 85%, 90%, 91%, 92%, 93%, 94%, 95%, 96%, 97%, 98%, 99%, or 100% identity to the sequence set forth in any one of SEQ ID NOs: 6-16.

6. (canceled)

7. The antibody or the antigen-binding fragment according to claim 1, wherein the single-domain antibody comprises an FR region in the VHH domain set forth in any one of SEQ ID NOs: 6-16; or
the single-domain antibody comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 6-16; the mutation may be selected from an insertion, a deletion, and/or a substitution; the substitution is preferably a substitution of conserved amino acids; or
the single-domain antibody comprises a sequence having at least 80%, 85%, 90%, 91%, 92%, 93%, 94%, 95%, 96%, 97%, 98%, 99%, or 100% identity to the FR region in the VHH domain set forth in any one of SEQ ID NOs: 6-16.

8. The antibody or the antigen-binding fragment according to claim 1, wherein the antibody or the antigen-binding fragment is: (1) a chimeric antibody or a fragment thereof, (2) a humanized antibody or a fragment thereof, or (3) a fully human antibody or a fragment thereof.

9. The antibody or the antigen-binding fragment according to claim 1, wherein the antibody or the antigen-binding fragment comprises or does not comprise an antibody heavy chain constant region; optionally, the antibody heavy chain constant region may be selected from human, Vicugna pacos, mouse, rat, rabbit, and sheep; or, the antibody heavy chain constant region may be selected from IgG, IgM, IgA, IgE, and IgD, and the IgG may be selected from IgG1, IgG2, IgG3, and IgG4; or, the antibody heavy chain constant region may be selected from an Fc region, a CH3 region, and an intact heavy chain constant region, and preferably, a human Fc region; preferably, the antibody or the antigen-binding fragment is a heavy chain antibody.

10. The antibody or the antigen-binding according to claim 1, wherein the antibody or the antigen-binding fragment is further conjugated to a therapeutic agent or a tracer; optionally, the therapeutic agent is selected from a radioisotope, a chemotherapeutic agent and an immunomodulator, and the tracer is selected from a radiocontrast medium, a paramagnetic ion, a metal, a fluorescent label, a chemiluminescent label, an ultrasound contrast agent, and a photosensitizer; optionally, the cytotoxic agent is selected from an alkaloid, methotrexate, doxorubicin, a taxane, and a toxin compound; the toxin compound is preferably selected from DM1, DM4, SN-38, MMAE, MMAF, duocarmycin, calicheamicin, and DX8951.

11. The antibody or the antigen-binding fragment according to claim 1, wherein the antibody or the antigen-binding fragment specifically binds to human and/or monkey CD5; optionally, the antibody or the antigen-binding fragment binds to human CD5 with a KD greater than 1.00E-6 M, 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M, 1.00E-10 M, 2.00E-10 M, 3.00E-10 M, 4.00E-10 M, 5.00E-10 M, 6.00E-10 M, 7.00E-10 M, 8.00E-10 M, 9.00E-10 M, 1.00E-11 M, or 1.00E-12 M.

12. A polypeptide, comprising the antibody or the antigen-binding fragment according to claim 1, wherein optionally, the polypeptide is further linked to an additional functional molecule; the additional functional molecules may be selected from one or more of a signal peptide, a protein tag or an additional antigen-binding molecule, and a cytokine;
optionally, the additional antigen-binding molecule specifically binds to an antigen other than CD5 or binds to a CD5 epitope different from that of the antibody or the antigen-binding fragment according to claim 1; optionally, the antigen other than CD5 may be selected from: CD3, preferably, CD38; CD16, preferably, CD16A; CD19; TGF-beta II receptor; NKG2D; CD40; 4-1BB; CD137 or CD19; EGFR; EGFRvIII; mesothelin; HER2; EphA2; Her3; EpCAM; MUC1; MUC16; CEA; Claudin18.2; folate receptor; Claudin6; WT1; NY-ESO-1; MAGE3; and ASGPR1 or CDH16;
optionally, the additional antigen-binding molecule is an antibody or antigen-binding fragment;
optionally, the cytokine may be selected from IL2, IL-6, IL-12, IL-15, IL-21, IFN, and TNF-alpha;
optionally, the polypeptide is a multispecific antigen-binding molecule, for example, bispecific, trispecific, or tetraspecific; more preferably, the multispecific antigen-binding molecule may be divalent, tetravalent, or hexavalent.

13-14. (canceled)

15. A chimeric antigen receptor (CAR), comprising an extracellular antigen-binding domain, a transmembrane domain, and an intracellular signaling domain, wherein the extracellular antigen-binding domain comprises the antibody or the antigen-binding fragment according to claim 1.

16. An immune effector cell expressing the chimeric antigen receptor according to claim 15, or comprising a nucleic acid fragment encoding the chimeric antigen receptor according to claim 15, wherein optionally, the immune effector cell is selected from a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell; optionally, the immune effector cell is an autoimmune effector cell or an allogeneic immune effector cell.

17. An isolated nucleic acid fragment encoding the antibody or the antigen-binding fragment according to claim 1.

18. A vector, comprising the nucleic acid fragment according to claim 17.

19. A host cell, comprising the vector according to claim 18, wherein optionally, the cell is a prokaryotic cell or a eukaryotic cell, such as a bacteria (Escherichia coli), a fungus (yeast), an insect cell, or a mammalian cell (a CHO cell or a 293T cell).

20-21. (canceled)

22. A pharmaceutical composition, comprising the antibody or the antigen-binding fragment according to claim 1, wherein optionally, the pharmaceutical composition further comprises a pharmaceutically acceptable carrier, a diluent, or an adjuvant; and optionally, the pharmaceutical composition further comprises an additional antineoplastic agent.

23. A pharmaceutical composition, comprising the antibody or the antigen-binding fragment according to claim 1, and any other active ingredients.

24. A method for the treatment of a disease, comprising administering an effective amount of the antibody or the antigen-binding fragment according to claim 1 to a subject, wherein the disease is selected from a tumor or a cancer and an autoimmune disease; the tumor or the cancer may be selected from a solid tumor and a hematologic tumor; the hematologic tumor may be selected from T-cell lymphoma, chronic lymphoma leukemia, cutaneous T-cell lymphoma, T-cell acute lymphocytic leukemia, and non-Hodgkin's lymphoma; the autoimmune disease may be selected from rheumatoid arthritis and graft-versus-host disease.

25-26. (canceled)

27. A kit, comprising the antibody or the antigen-binding fragment according to claim 1.

28. A method for detecting CD5 expression in a biological sample, comprising contacting the biological sample with the antibody or the antigen-binding fragment according to claim 1 in a condition allowing formation of a complex between the antibody or the antigen-binding fragment according to claim 1 and CD5, wherein preferably, the method further comprises detecting the formation of the complex, and indicating the presence or an expression level of CD5 in the sample.

29. (canceled)


SEQUENCE LISTING (121 sequences)

SEQ ID NO: 1 - DNA, 18 bp, Artificial Sequence
  Synthetic polynucleotide
cttggtggtcctggctgc

SEQ ID NO: 2 - DNA, 23 bp, Artificial Sequence
  Synthetic polynucleotide
ggtacgtgctgttgaactgttcc

SEQ ID NO: 3 - DNA, 47 bp, Artificial Sequence
  Synthetic polynucleotide
catgccatgactgtggcccaggcggcccagktgcagctcgtggagtc

SEQ ID NO: 4 - DNA, 51 bp, Artificial Sequence
  Synthetic polynucleotide
catgccatgactcgcggccggcctggccatgggggtcttcgctgtggtgcg

SEQ ID NO: 5 - DNA, 52 bp, Artificial Sequence
  Synthetic polynucleotide
catgccatgactcgcggccggcctggccgtcttgtggttttggtgtcttggg

SEQ ID NO: 6 - protein, 123 aa, Artificial Sequence
  Synthetic polypeptide
QLQLVESGGGLVQPGGSLRLSCAASGFTFSSYAMSWYRQAPGKERELVAAITSAGGSTTYADSVKDRFTISRDNAKNTVSLQMNSLKPEDTAVYYCNAYCTGYVCHEGGYDYWGQGTQVTVSS

SEQ ID NO: 7 - protein, 121 aa, Artificial Sequence
  Synthetic polypeptide
QVQLVESGGGLVQAGGSLRLSCAASGSIFSISVMGWYRQAPGKERETVAVISSGGSTYYADSVKGRFTISRDNAKNTLYLQMNSLKPEDTAVYYCNADFETASGSPLYESWGQGTQVTVSS

SEQ ID NO: 8 - protein, 118 aa, Artificial Sequence
  Synthetic polypeptide
EVQVVESGGGLVQAGGSLRLSCAASGSIFSINLLGWYRQAPGKERESVAILSSGGSTYYADSVKGRFTISRDNVKNMLYLQMNSLKPEDTAVYYCNAVGGIATLRDYWGQGTQVTVSS

SEQ ID NO: 9 - protein, 134 aa, Artificial Sequence
  Synthetic polypeptide
EVQVVESGGGLVQPGGSLRLSCAASGFTLDYYAIGWFRQAPGKEREGLSCISTSGGSTNYADSVKGRFTISRDNAKNTVYLQMNSLKPEDTAVYYCAASSFWDCAVVGTGSPKTGHLESVFGSWGQGTQVTVSS

SEQ ID NO: 10 - protein, 134 aa, Artificial Sequence
  Synthetic polypeptide
QVQLVESGGGLVQPGGSLRLSCAASGFTWDYYAIGWFRQAPGKEREGVSCIDSSGGSINYLDSVKGRFTVSRDNAKNTVYLQMDSLKPEDTAIYYCAASSYWDCAVPDTGSPKTGHLESYFGSWGQGTQVIVSS

SEQ ID NO: 11 - protein, 122 aa, Artificial Sequence
  Synthetic polypeptide
EVQVVESGGGLVQAGGSLRLSCAASGDTICISAMYWYRQAPGKAREMVAAITSGGSTYYEDSVMGRFTIFRENAKNTVYLQMNSLKPEDTAVYYCNADIAGHNCSGYLKEYWGQGTQVTVSA

SEQ ID NO: 12 - protein, 129 aa, Artificial Sequence
  Synthetic polypeptide
QVQLVESGGGLVQPGGSLRLSCAASGFSLDYYAIGWFRQAPGKEREGVSYISPSDGRTYYADSVKGRFTISRDNAKNTVYLQMNSLKPEDTAVYYCATDWGSTSFDYGLKLSPTQYGYWGQGTQVTVSS

SEQ ID NO: 13 - protein, 127 aa, Artificial Sequence
  Synthetic polypeptide
QVQLVESGGGLVQAGGSLRLSCAVSGRTLSTVVVGWFRQTPGREREFVAVSRSGGATGYADSVKDRFTISRDNAKNTVALQMNSLKPEDTAVYYCAGRGSGLYGSSWYNQIQEYDGWGQGTQVTVSS

SEQ ID NO: 14 - protein, 121 aa, Artificial Sequence
  Synthetic polypeptide
EVQLVESGGGLVQAGGSLRLSCAASESTFSITAMGWYRQAPGKQRELVAAITSPGGSTNYADSVKGRFTISRDNAKNTMLLQMNSLKPEDTAVYYCNADINHGSGYYTVSWGQGTQVTVSS

SEQ ID NO: 15 - protein, 129 aa, Artificial Sequence
  Synthetic polypeptide
EVQLVESGGGLVQPGGSLRLSCAASGFSLDYYAIGWFRQAPGKEREGVSYISASDGSTYYADSVKGRFTISRDNAKNTVYLQMNSLKPEDTAVYYCATDWGSGSFNYGLRLSPTQYDYWGQGTQVTVSS

SEQ ID NO: 16 - protein, 120 aa, Artificial Sequence
  Synthetic polypeptide
EVQVVESGGGLVQAGGSLRLSCAASGSIFSINAMGWYRQAPGKEREVVAAISNGGNTYYEDSVMGRFTISRDNAKGTLYLQMNGLKPEDTAVYYCNADTVVGGGLLTEVWGQGTLVTVSS

SEQ ID NO: 17 - protein, 8 aa, Artificial Sequence
  Synthetic polypeptide
GFTFSSYA

SEQ ID NO: 18 - protein, 8 aa, Artificial Sequence
  Synthetic polypeptide
ITSAGGST

SEQ ID NO: 19 - protein, 16 aa, Artificial Sequence
  Synthetic polypeptide
NAYCTGYVCHEGGYDY

SEQ ID NO: 20 - protein, 5 aa, Artificial Sequence
  Synthetic polypeptide
SYAMS

SEQ ID NO: 21 - protein, 17 aa, Artificial Sequence
  Synthetic polypeptide
AITSAGGSTTYADSVKD

SEQ ID NO: 22 - protein, 14 aa, Artificial Sequence
  Synthetic polypeptide
YCTGYVCHEGGYDY

SEQ ID NO: 23 - protein, 7 aa, Artificial Sequence
  Synthetic polypeptide
GFTFSSY

SEQ ID NO: 24 - protein, 6 aa, Artificial Sequence
  Synthetic polypeptide
TSAGGS

SEQ ID NO: 25 - protein, 14 aa, Artificial Sequence
  Synthetic polypeptide
YCTGYVCHEGGYDY

SEQ ID NO: 26 - protein, 8 aa, Artificial Sequence
  Synthetic polypeptide
GSIFSISV

SEQ ID NO: 27 - protein, 7 aa, Artificial Sequence
  Synthetic polypeptide
ISSGGST

SEQ ID NO: 28 - protein, 15 aa, Artificial Sequence
  Synthetic polypeptide
NADFETASGSPLYES

SEQ ID NO: 29 - protein, 5 aa, Artificial Sequence
  Synthetic polypeptide
ISVMG

SEQ ID NO: 30 - protein, 16 aa, Artificial Sequence
  Synthetic polypeptide
VISSGGSTYYADSVKG

SEQ ID NO: 31 - protein, 13 aa, Artificial Sequence
  Synthetic polypeptide
DFETASGSPLYES

SEQ ID NO: 32 - protein, 7 aa, Artificial Sequence
  Synthetic polypeptide
GSIFSIS

SEQ ID NO: 33 - protein, 5 aa, Artificial Sequence
  Synthetic polypeptide
SSGGS

SEQ ID NO: 34 - protein, 13 aa, Artificial Sequence
  Synthetic polypeptide
DFETASGSPLYES

SEQ ID NO: 35 - protein, 8 aa, Artificial Sequence
  Synthetic polypeptide
GSIFSINL

SEQ ID NO: 36 - protein, 7 aa, Artificial Sequence
  Synthetic polypeptide
LSSGGST

SEQ ID NO: 37 - protein, 12 aa, Artificial Sequence
  Synthetic polypeptide
NAVGGIATLRDY

SEQ ID NO: 38 - protein, 5 aa, Artificial Sequence
  Synthetic polypeptide
INLLG

SEQ ID NO: 39 - protein, 16 aa, Artificial Sequence
  Synthetic polypeptide
ILSSGGSTYYADSVKG

SEQ ID NO: 40 - protein, 10 aa, Artificial Sequence
  Synthetic polypeptide
VGGIATLRDY

SEQ ID NO: 41 - protein, 7 aa, Artificial Sequence
  Synthetic polypeptide
GSIFSIN

SEQ ID NO: 42 - protein, 5 aa, Artificial Sequence
  Synthetic polypeptide
SSGGS

SEQ ID NO: 43 - protein, 10 aa, Artificial Sequence
  Synthetic polypeptide
VGGIATLRDY

SEQ ID NO: 44 - protein, 8 aa, Artificial Sequence
  Synthetic polypeptide
GFTLDYYA

SEQ ID NO: 45 - protein, 8 aa, Artificial Sequence
  Synthetic polypeptide
ISTSGGST

SEQ ID NO: 46 - protein, 27 aa, Artificial Sequence
  Synthetic polypeptide
AASSFWDCAVVGTGSPKTGHLESVFGS

SEQ ID NO: 47 - protein, 5 aa, Artificial Sequence
  Synthetic polypeptide
YYAIG

SEQ ID NO: 48 - protein, 17 aa, Artificial Sequence
  Synthetic polypeptide
CISTSGGSTNYADSVKG

SEQ ID NO: 49 - protein, 25 aa, Artificial Sequence
  Synthetic polypeptide
SSFWDCAVVGTGSPKTGHLESVFGS

SEQ ID NO: 50 - protein, 7 aa, Artificial Sequence
  Synthetic polypeptide
GFTLDYY

SEQ ID NO: 51 - protein, 6 aa, Artificial Sequence
  Synthetic polypeptide
STSGGS

SEQ ID NO: 52 - protein, 25 aa, Artificial Sequence
  Synthetic polypeptide
SSFWDCAVVGTGSPKTGHLESVFGS

SEQ ID NO: 53 - protein, 8 aa, Artificial Sequence
  Synthetic polypeptide
GFTWDYYA

SEQ ID NO: 54 - protein, 8 aa, Artificial Sequence
  Synthetic polypeptide
IDSSGGSI

SEQ ID NO: 55 - protein, 27 aa, Artificial Sequence
  Synthetic polypeptide
AASSYWDCAVPDTGSPKTGHLESYFGS

SEQ ID NO: 56 - protein, 5 aa, Artificial Sequence
  Synthetic polypeptide
YYAIG

SEQ ID NO: 57 - protein, 17 aa, Artificial Sequence
  Synthetic polypeptide
CIDSSGGSINYLDSVKG

SEQ ID NO: 58 - protein, 25 aa, Artificial Sequence
  Synthetic polypeptide
SSYWDCAVPDTGSPKTGHLESYFGS

SEQ ID NO: 59 - protein, 7 aa, Artificial Sequence
  Synthetic polypeptide
GFTWDYY

SEQ ID NO: 60 - protein, 6 aa, Artificial Sequence
  Synthetic polypeptide
DSSGGS

SEQ ID NO: 61 - protein, 25 aa, Artificial Sequence
  Synthetic polypeptide
SSYWDCAVPDTGSPKTGHLESYFGS

SEQ ID NO: 62 - protein, 8 aa, Artificial Sequence
  Synthetic polypeptide
GDTICISA

SEQ ID NO: 63 - protein, 7 aa, Artificial Sequence
  Synthetic polypeptide
ITSGGST

SEQ ID NO: 64 - protein, 16 aa, Artificial Sequence
  Synthetic polypeptide
NADIAGHNCSGYLKEY

SEQ ID NO: 65 - protein, 5 aa, Artificial Sequence
  Synthetic polypeptide
ISAMY

SEQ ID NO: 66 - protein, 16 aa, Artificial Sequence
  Synthetic polypeptide
AITSGGSTYYEDSVMG

SEQ ID NO: 67 - protein, 14 aa, Artificial Sequence
  Synthetic polypeptide
DIAGHNCSGYLKEY

SEQ ID NO: 68 - protein, 7 aa, Artificial Sequence
  Synthetic polypeptide
GDTICIS

SEQ ID NO: 69 - protein, 5 aa, Artificial Sequence
  Synthetic polypeptide
TSGGS

SEQ ID NO: 70 - protein, 14 aa, Artificial Sequence
  Synthetic polypeptide
DIAGHNCSGYLKEY

SEQ ID NO: 71 - protein, 8 aa, Artificial Sequence
  Synthetic polypeptide
GFSLDYYA

SEQ ID NO: 72 - protein, 8 aa, Artificial Sequence
  Synthetic polypeptide
ISPSDGRT

SEQ ID NO: 73 - protein, 22 aa, Artificial Sequence
  Synthetic polypeptide
ATDWGSTSFDYGLKLSPTQYGY

SEQ ID NO: 74 - protein, 5 aa, Artificial Sequence
  Synthetic polypeptide
YYAIG

SEQ ID NO: 75 - protein, 17 aa, Artificial Sequence
  Synthetic polypeptide
YISPSDGRTYYADSVKG

SEQ ID NO: 76 - protein, 20 aa, Artificial Sequence
  Synthetic polypeptide
DWGSTSFDYGLKLSPTQYGY

SEQ ID NO: 77 - protein, 7 aa, Artificial Sequence
  Synthetic polypeptide
GFSLDYY

SEQ ID NO: 78 - protein, 6 aa, Artificial Sequence
  Synthetic polypeptide
SPSDGR

SEQ ID NO: 79 - protein, 20 aa, Artificial Sequence
  Synthetic polypeptide
DWGSTSFDYGLKLSPTQYGY

SEQ ID NO: 80 - protein, 8 aa, Artificial Sequence
  Synthetic polypeptide
GRTLSTVV

SEQ ID NO: 81 - protein, 8 aa, Artificial Sequence
  Synthetic polypeptide
VSRSGGAT

SEQ ID NO: 82 - protein, 21 aa, Artificial Sequence
  Synthetic polypeptide
AGRGSGLYGSSWYNQIQEYDG

SEQ ID NO: 83 - protein, 5 aa, Artificial Sequence
  Synthetic polypeptide
TVVVG

SEQ ID NO: 84 - protein, 16 aa, Artificial Sequence
  Synthetic polypeptide
VSRSGGATGYADSVKD

SEQ ID NO: 85 - protein, 19 aa, Artificial Sequence
  Synthetic polypeptide
RGSGLYGSSWYNQIQEYDG

SEQ ID NO: 86 - protein, 7 aa, Artificial Sequence
  Synthetic polypeptide
GRTLSTV

SEQ ID NO: 87 - protein, 5 aa, Artificial Sequence
  Synthetic polypeptide
RSGGA

SEQ ID NO: 88 - protein, 19 aa, Artificial Sequence
  Synthetic polypeptide
RGSGLYGSSWYNQIQEYDG

SEQ ID NO: 89 - protein, 8 aa, Artificial Sequence
  Synthetic polypeptide
ESTFSITA

SEQ ID NO: 90 - protein, 8 aa, Artificial Sequence
  Synthetic polypeptide
ITSPGGST

SEQ ID NO: 91 - protein, 14 aa, Artificial Sequence
  Synthetic polypeptide
NADINHGSGYYTVS

SEQ ID NO: 92 - protein, 5 aa, Artificial Sequence
  Synthetic polypeptide
ITAMG

SEQ ID NO: 93 - protein, 17 aa, Artificial Sequence
  Synthetic polypeptide
AITSPGGSTNYADSVKG

SEQ ID NO: 94 - protein, 12 aa, Artificial Sequence
  Synthetic polypeptide
DINHGSGYYTVS

SEQ ID NO: 95 - protein, 7 aa, Artificial Sequence
  Synthetic polypeptide
ESTFSIT

SEQ ID NO: 96 - protein, 6 aa, Artificial Sequence
  Synthetic polypeptide
TSPGGS

SEQ ID NO: 97 - protein, 12 aa, Artificial Sequence
  Synthetic polypeptide
DINHGSGYYTVS

SEQ ID NO: 98 - protein, 8 aa, Artificial Sequence
  Synthetic polypeptide
GFSLDYYA

SEQ ID NO: 99 - protein, 8 aa, Artificial Sequence
  Synthetic polypeptide
ISASDGST

SEQ ID NO: 100 - protein, 22 aa, Artificial Sequence
  Synthetic polypeptide
ATDWGSGSFNYGLRLSPTQYDY

SEQ ID NO: 101 - protein, 5 aa, Artificial Sequence
  Synthetic polypeptide
YYAIG

SEQ ID NO: 102 - protein, 17 aa, Artificial Sequence
  Synthetic polypeptide
YISASDGSTYYADSVKG

SEQ ID NO: 103 - protein, 20 aa, Artificial Sequence
  Synthetic polypeptide
DWGSGSFNYGLRLSPTQYDY

SEQ ID NO: 104 - protein, 7 aa, Artificial Sequence
  Synthetic polypeptide
GFSLDYY

SEQ ID NO: 105 - protein, 6 aa, Artificial Sequence
  Synthetic polypeptide
SASDGS

SEQ ID NO: 106 - protein, 20 aa, Artificial Sequence
  Synthetic polypeptide
DWGSGSFNYGLRLSPTQYDY

SEQ ID NO: 107 - protein, 8 aa, Artificial Sequence
  Synthetic polypeptide
GSIFSINA

SEQ ID NO: 108 - protein, 7 aa, Artificial Sequence
  Synthetic polypeptide
ISNGGNT

SEQ ID NO: 109 - protein, 14 aa, Artificial Sequence
  Synthetic polypeptide
NADTVVGGGLLTEV

SEQ ID NO: 110 - protein, 5 aa, Artificial Sequence
  Synthetic polypeptide
INAMG

SEQ ID NO: 111 - protein, 16 aa, Artificial Sequence
  Synthetic polypeptide
AISNGGNTYYEDSVMG

SEQ ID NO: 112 - protein, 12 aa, Artificial Sequence
  Synthetic polypeptide
DTVVGGGLLTEV

SEQ ID NO: 113 - protein, 7 aa, Artificial Sequence
  Synthetic polypeptide
GSIFSIN

SEQ ID NO: 114 - protein, 5 aa, Artificial Sequence
  Synthetic polypeptide
SNGGN

SEQ ID NO: 115 - protein, 12 aa, Artificial Sequence
  Synthetic polypeptide
DTVVGGGLLTEV

SEQ ID NO: 116 - protein, 232 aa, Artificial Sequence
  Synthetic polypeptide
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 118 aa, Artificial Sequence
  Synthetic polypeptide
NIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSS

SEQ ID NO: 118 - protein, 107 aa, Artificial Sequence
  Synthetic polypeptide
DIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMK

SEQ ID NO: 119 - protein, 472 aa, Artificial Sequence
  Synthetic polypeptide
NIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSGGGGSGGGGSGGGGSDIKMTQSPSSMYASLGERVTITCKASQDINSYLSWFHHKPGKSPKTLIYRANRLVDGVPSRFSGSGSGQDYSLTISSLDYEDMGIYYCQQYDESPWTFGGGTKLEMKEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 493 aa, Artificial Sequence
  Synthetic polypeptide
MGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 121 - protein, 499 aa, Artificial Sequence
  Synthetic polypeptide
MPMRSPQPLATLYLLGMLVASCLGRLSWDDPDFQTRLTRSNSRCQGQLEVYIKYGWHMVCSQSWGRSSNQWEDPNQASKVCQRLNCGVPLSLGPFLITDRHQSQITCYGRLGSFSNCSHSGRDVCRPLGLTCLEPQTTTPPPTRPPPTTTPEPTAPPRLQLVAQAAGRHCAGVVEFYSGSLGGTISYEAQDKAQDKTQVLENFLCSSLQCGSFLKHLPETEAATAQDPGELREHQPLPIQWTIRNSSCTSLEHCFRKIKPRNSGQVLALLCSGFQPKVQSRLVGGSSICEGTVEVRQGAQWAALCDSSSAKSSLRWEEVCQEQQCGSFNSYQALDAGDPTSRGLSCPHQKLSQCHELTERKSYCKKVFVTCQDPNPAGPAAGTVASIILALVLLGVLLVVCGPLAYKKLVKKFRQKKQRQWIGPTGMNQNMSFHRNHTATVRSHVENPTASHVDNEYSQPPRNSRLSAYPALEGALHRSSTQPDNSSDSDYDLHGAQRL